(19) United States
(12) Patent Application Publication
(10) Pub. No.: US 2018/0273961 A1
(43) Pub. Date: Sep. 27, 2018

(54) A CRISPR/Cas9 SYSTEM FOR HIGH EFFICIENT SITE-DIRECTED ALTERING OF PLANT GENOMES

(71) Applicant: Institute of Genetics and Developmental Biology, Chinese Academy of Sciences, Beijing (CN)

(72) Inventors: Qi Xie, Beijing (CN); Liuhua Yan, Beijing (CN); Shaowei Wei, Beijing (CN); Weicai Yang, Beijing (CN); Hongju Li, Beijing (CN)

(21) Appl. No.: 15/764,217

(22) PCT Filed: Sep. 28, 2016
(86) PCT No.: PCT/CN2016/100533

§ 371 Date: Mar. 28, 2018

(30) Foreign Application Priority Data
Sep. 29, 2015 (CN) 201510631450.5

(51) Int. Cl. C12N 15/82; C12N 15/113

(52) U.S. Cl. CPC C12N 15/8213; C12N 2310/20; C12N 15/8261; C12N 15/113

(57) ABSTRACT

Cassettes comprising a YAO promoter operably linked to at least one nucleotide sequence encoding a nuclease, vectors comprising the same are provided. A system for altering a plant genome comprising a nucleotide sequence encoding a nuclease operably linked to a YAO promoter and a method to alter the target nucleic acid molecule by using the system are provided. Plants, progeny and seeds thereof having such altered target nucleic acid molecules are also provided.

### REFERENCE TO RELATED APPLICATIONS

This application claims priority to previously filed and co-pending application CN105177038, filed Sep. 29, 2015, the contents of which are incorporated herein by reference in its entirety.

### SEQUENCE LISTING

The instant application contains a Sequence Listing which has been submitted in ASCII format and is hereby incorporated by reference in its entirety. Said Sequence Listing, created on Sep. 26, 2016, is named P12040WO00_SL.txt and is 105,189 bytes in size.

### TECHNICAL FIELD

The present invention relates to the field of biotechnology, particularly a CRISPR/Cas9 system for high efficient site-directed altering of plant genomes.

### BACKGROUND

The realization of high efficient, site-directed altering for plant genomes is of great significance to study the functions of plant genes. At present, gene modification techniques, such as zinc finger nucleases (ZFNs), transcription activator-like effector nucleases (TALEN), and CRISPR/Cas9 etc., have been widely used in scientific research, wherein the CRISPR/Cas9 technique is a recently developed gene modification technique. The CRISPR/Cas system is an acquired immune system presently discovered which exists in most bacteria and all archaea to eliminate extraneous plastids or phages, and to leave extraneous gene fragments in autologous genomes as “memories”. Different forms of deletions or insertions have been created at target fragments by editing organism genomes with a CRISPR/Cas9 system, which has been successfully used in organisms such as Homo sapiens cell lines, Danio rerio, Rattus norvegicus, Mus musculus, Drosophila melanogaster etc. In the field of plants, this technique has also been used in plants such as Arabidopsis thaliana, Oryza sativa L., Zea mays L., Nicotiana tabacum, Lycopersicon esculentum etc., but the editing efficiency of the existing CRISPR/Cas9 system is low.

At present, the promoters used for driving nucleases in these systems, such as the Cas9 gene expressionor FokI gene expression are mostly are CMV 35S promoter and Ubiquitin promoter, but previous studies have demonstrated that, the editing efficiencies of Cas9 to plant genomes driven by the both are low. It can be seen that, for improving the editing efficiencies, it is especially important to select suitable promoters for driving the expression of Cas9 gene.

### SUMMARY OF THE INVENTION

Increased frequency of gene altering is provided by use of a YAO promoter. When used with a gene editing system such as CRISPR/Cas9, TALEN or Zinc finger nucleases, the frequency of gene editing is increased compared to use of a promoter that is not the YAO promoter and in particular compared to using the 35S promoter. In one embodiment the YAO promoter is operably linked with a nucleic acid molecule that encodes a Cas9 or FokI polypeptide. Gene editing frequency is increased to at least 75% or more and up to 90%, 95% or more. The frequency of gene editing of a targeted nucleic acid molecule is at least five times, 18 times or higher than when using a 35S promoter. The increased gene frequency is also provided in progeny of a plant into which a cassette is introduced comprising the YAO promoter driving a nuclease such as the Cas9 or FokI nucleic acid molecule. Cassettes, vector, edited plants and cells are also provided.

### BRIEF DESCRIPTION OF THE DRAWINGS

FIGS. 1A and 1B is a diagram showing structure of the CRISPR/Cas9 binary vectors for Arabidopsis transformation. The hSpCas9 cassette is driven by the 35S (see FIG. 1A) or YAO (FIG. 1B) promoter, while sgRNA is controlled by the AtU6-26 promoter. NLS refers to the nuclear localization sequence.

FIG. 2 is a gel showing RFLP detect the site-directed editing effects of 35S:Cas9/AtU6-26-sgRNA system and pYAO:Cas9/AtU6-26-sgRNA system on endogenous gene BRI1 of Arabidopsis thaliana. Here, M is a DNA Marker; Lanes 1-23 in FIG. 2A are electrophoresis results of PCR products of T1 generation Arabidopsis thaliana introduced with 35S:Cas9/AtU6-26-sgRNA system after EcoR V enzyme cleavage, Lanes 1-21 in FIG. 2B are electrophoresis results of PCR products of T1 generation Arabidopsis thaliana introduced with pYAO:Cas9/AtU6-26-sgRNA system after EcoR V enzyme cleavage; and Col-0 is electrophoresis result of PCR products of wild type Arabidopsis thaliana after EcoR V enzyme cleavage.

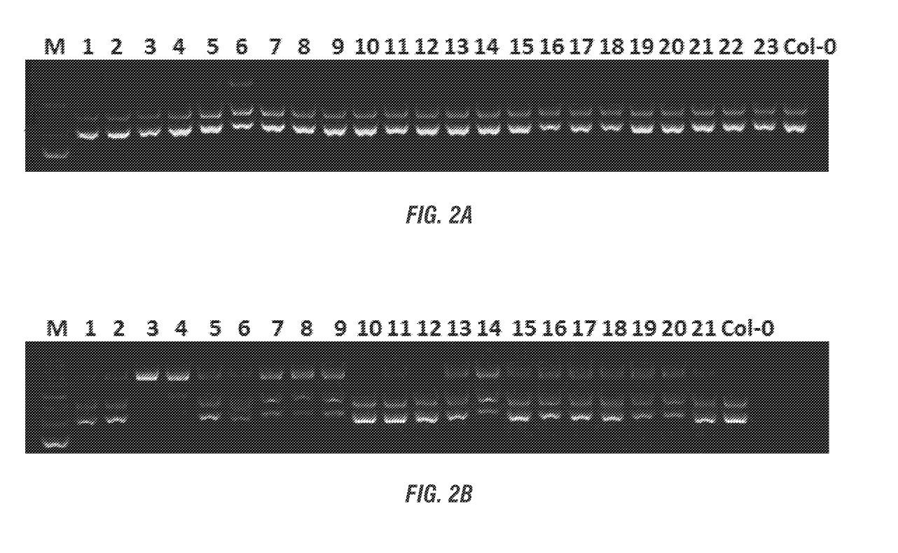

FIG. 3A-C are graphs showing sequencing analysis for site-directed editing effects of 35S:Cas9/AtU6-26-sgRNA system and pYAO:Cas9/AtU6-26-sgRNA system on endogenous gene BRI1 of T1 generation Arabidopsis thaliana. Here, FIG. 3A is a peak profile of sequencing for PCR products of 35S:hSpCas9-BRI1-sgRNA system vs. 35S-6-T1; FIG. 3B is a peak profile of sequencing for PCR products of pYAO:hSpCas9-BRI1-sgRNA system vs. pYAO-16-T1; FIG. 3C is a peak profile of sequencing for PCR products of pYAO:hSpCas9-BRI1-sgRNA system vs. pYAO-3-T1

FIG. 4A FIG. 4A shows editing forms of 35S-6-T1 and pYAO-16-T1 at target sites of BRI1 gene (SEQ ID NOS 75-77, respectively, in order of appearance); and FIG. 4B shows editing forms of pYAO-3-T1 at target sites of BRI1 gene (SEQ ID NOS 75, 78, 79, 77 and 80, respectively, in order of appearance); WT represents the nucleotide sequences of wild-type Arabidopsis thaliana at the target sites, “D” represents the sequences subjected to deletion mutations, “+” represents the sequences subjected to insertion mutations, and the numbers behind “D/+” represent the amount of deleted or inserted nucleotides.

FIG. 5 shows representative sequences of several mutant alleles of BRI1 identified from the pYAO:hSpCas9-BRI1-sgRNA T1 transgenic plant line 4 and line 21 (SEQ ID NOS 81-86, 83 and 87, respectively, in order of appearance). The wild-type sequence is shown at the top with the PAM sequence in bold.

FIG. 6A is a gel showing RFLP analysis of genomic DNA from the pYAO:hSpCas9-PDS3-sgRNA T1 plants. FIG. 6B shows representative sequences of several mutant alleles of PDS3 identified from a pYAO:hSpCas9-PDS3-sgRNA T1 transgenic plant (SEQ ID NOS 88-96, 91, 94, 92, 97, 91 and 98, respectively, in order of appearance). The PAM sequence is shown in bold. The target sequence is in the frame.

FIGS. 7A and 7B show representative sequences of several mutant alleles of SlPDS3 and SlGLK1 identified from the pYAO:Cas9-SlPDS3 (SEQ ID NOS 99-103, 100, 104, 103 and 105, respectively, in order of appearance) (FIG. 7A) and pYAO:Cas9-SlGLK1 (SEQ ID NOS 106-111, 60-62, 108, 109, 112, 111, 113, 114, and 69-71, respectively, in order of appearance) (FIG. 7B) T1 transgenic plants. The wild-type sequence is shown at the top (SEQ ID NO: 99 in FIG. 7A and SEQ ID NO: 106 in FIG. 7B) with the PAM sequence highlighted in bold. The target sequence is in the frame.

FIGS. 8A and 8B are diagrams of construct prepared for use in zinc finger process (FIG. 8A) and in a TALEN gene altering system (FIG. 8B) wherein the YAO promoter is driving a first and second zinc finger polypeptide (ZFP) or expression of a first and second transcription activator-like effector (TALE) repeat sequence, where FokI represents the FokI endonuclease sequence.

FIG. 9 shows results of alignment of the Arabidopsis and Zea mays YAO polypeptide, with the consensus sequence shown below.

FIG. 10 is a graphic representation of regions of the Arabidopsis YAO promoter and the Zea mays YAO promoter.

### DESCRIPTION

The technical problem sought to be solved by the present invention is to provide a method for high efficient site-directed editing of plant genomes.

In order to solve the above technical problem, the present invention provides an expression cassette (here for convenience referred to as expression cassette I) containing a promoter pYAO. In the expression cassette, the expression of the coding gene of Cas9 nuclease is initiated by the promoter pYAO. The promoter pYAO can be following (a1) or (a2) or (a3) or (a4) or (a5):

(a1) a DNA molecule shown by Sites 1-1012 (1-982 bp 5′ terminal promoter region+30 bp Yao ORF) (SEQ ID NO: 2) from 5′ terminal end in SEQ ID NO: 1;
(a2) a DNA molecule having 50%, 55%, 65%, 75%, 80%, 85%, 90%, 95% and amounts in-between, or higher identity with the nucleotide sequence defined by (a1), and having promoter function; or
(a3) a DNA molecule comprising a regulatory region of a YAO gene having promoter function;
(a4) a DNA molecule hybridizing with the nucleotide sequences defined by (a1) or (a2) or (a3) under stringent condition, and having promoter function and in particular promoter function which provides for increased gene editing as described herein; or
(a5) a functional fragment of any of (a1)-(a4).

As discussed further herein, the promoter described here is useful in increasing the frequency of genome editing and in an embodiment when using a CRISPR/Cas9 gene editing process. The YAO promoter in an embodiment is used to transcribe a Cas9 nuclease when editing genes with the CRISPR/Cas9 process. The frequency of gene editing is up to 50%, at least 60%, 65%, 70%, 75%, 80%, 85%, 90%, 95% or more and percentages in between. When referring to increasing the frequency of gene editing it is meant that the frequency of inserting, deleting or modifying a targeted region of a eukaryotic or prokaryotic gene. This frequency is increased when using the CRISPR/Cas9 gene editing process compared to the frequency of genome editing when not using the YAO promoter, and in particular compared to use with the 35S promoter. The increase in frequency of gene editing can be twice, three times, four times, five times, up to 18 times or more than when using 35S promoter. Furthermore, progeny of plants into which the expression cassettes described are introduced are shown to inherit the higher frequency of genome editing associated with the YAO promoter. In an embodiment at least 75% of said progeny segregate having said edited target sequence.

The YAO gene encodes a nucleolar protein having seven WD repeats. It has been shown to have a role in cell division regulation during early embryogenesis in plants. Li et al. (2010) “YAO is a nucleolar WD40-repeat protein critical for embryogenesis and gametogenesis in Arabidopsis” BMC Plant Biology 10:169. The promoter is preferentially expressed in tissues which are undergoing active cell division, including shoot apical and root meristem and expresses at high levels in embryo sac, embryo, endosperm and pollen. An embodiment provides plant genomes can be highly efficiently edited using the YAO gene promoter and in an embodiment when expressed during plant gametophytic and/or early embryo development. When referring to a YAO promoter is meant to include a regulatory region of a YAO gene which encodes the YAO polypeptide as described, including for example a polypeptide encoded by SEQ ID NO: 1 and any variants which produce the YAO nucleolar protein having seven WD repeats and which retain the property of increased frequency of gene editing as described herein. Examples of the YAO amino acid encoded are found at Mayer et al. “WD40-repeats containing protein YAOZHE (Arabidopsis thaliana) GenBank Ref No. NP_192450 (January 2014) and at Mayer et al. Nature 402 (6763) 769-777 (1999) and Zapata et al, YAO (Arabidopsis thaliana) GenBank Ref No. OAP00198 (Mar. 14, 2016)

The promoter can be used in any plant species, including, for example, a monocotyledonous plant, including but not limited to wheat, rye, rice, oat, barley, turfgrass, sorghum, millet or sugarcane. Alternatively, the plant may be a dicotyledonous plant, including but not limited to tobacco, tomato, potato, soybean, cotton, canola, sunflower or alfalfa. Promoters from one species such as maize promoters have been used repeatedly to drive expression of genes in other non-maize plants, including tobacco (Yang and Russell (1990) “Maize sucrose synthase-1 promoter drives phloem cell-specific expression of GUS gene in transgenic tobacco plants” Proc. Natl. Acad. Sci. USA 87, 4144-4148; Geffers et al., (2000) “Anaerobiosis-specific interaction of tobacco nuclear factors with cis-regulatory sequences in the maize GapC4 promoter” Plant Mol. Biol. 43, 11-21; Vilardell et al., (1991) “Regulation of the maize rab 17 gene promoter in transgenic heterologous systems” Plant Mol. Biol. 17, 985-993), cultured rice cells (Vilardell et al. (1991), supra), wheat (Oldach et al., (2001) “Heterologous expression of genes mediating enhanced fungal resistance in transgenic wheat” Mol. Plant Microbe Interact. 14, 832-838; Brinch-Pedersen et al., (2003) “Concerted action of endogenous and heterologous phytase on phytic acid degradation in seed of transgenic wheat (Triticum aestivum L.)” Transgenic Res. 12, 649-659), rice (Cornejo et al., (1993) “Activity of a maize ubiquitin promoter in transgenic rice” Plant Mol. Biol. 23, 567-581; Takimoto et al., (1994) “Non-systemic expression of a stress-response maize polyubiquitin gene (Ubi-1) in transgenic rice plants” Plant Mol. Biol. 26, 1007-1012), sunflower (Roussell et al., (1988) “Deletion of DNA sequences flanking an Mr 19,000 zein gene reduces its transcriptional activity in heterologous plant tissues” Mol. Gen. Genet. 211, 202-209) and protoplasts of carrot (Roussell et al., 1988, supra).

The term plant or plant material or plant part is used broadly herein to include any plant at any stage of development, or to part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or aggregate of cells such as a friable callus, or a cultured cell, or can be part of a higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered a plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like. The tissue culture will preferably be capable of regenerating plants. Preferably, the regenerable cells in such tissue cultures will be embryos, protoplasts, meristematic cells, callus, pollen, leaves, anthers, roots, root tips, silk, flowers, kernels, ears, cobs, husks or stalks. Still further, provided are plants regenerated from the tissue cultures of the invention.

The nucleic acid molecules and polypeptides can be used to isolate corresponding sequences from other organisms, particularly other plants, or to synthesize synthetic sequences. In this manner, methods such as polymerase chain reaction (PCR), hybridization, synthetic gene construction and the like can be used to identify or generate such sequences based on their sequence homology to the sequences set forth herein. Sequences identified, isolated or constructed based on their sequence identity to the whole of or any portion of the sequences set forth is encompassed by the products and processes. Synthesis of sequences suitably employed can be effected by means of mutually priming long oligonucleotides. See for example, Wosnick et al. (1987) Gene 60:115. In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed (Sambrook, J., Fritsch, E. F. and Maniatis, T. (2001) Molecular Cloning: A Laboratory Manual, 3^{rd} Edition. Cold Spring Harbor Laboratory Press, Plainview, N. Y; Innis, M., Gelfand, D. and Sninsky, J. (1995) PCR Strategies. Academic Press, New York; Innis, M., Gelfand, D. and Sninsky, J. (1999) PCR Applications: Protocols for Functional Genomics, Academic Press, New York. Moreover, techniques which employ the PCR reaction permit the synthesis of genes as large as 1.8 kilobases in length. See Adang et al. (1993) Plant Molec. Biol. 21 (6):1131-45) and Bambot et al. (1993) PCR Methods and Applications 2:266-71. Known methods of PCR include, but are not limited to, methods using paired primers, nested primers, degenerate primers, gene-specific primers, vector-specific primers, partially-mismatched primers, and the like. In addition, genes can readily be synthesized by conventional automated techniques.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledons or dicotyledons as these preferences have been shown to differ (Murray et al. Nucl. Acids Res. 17:477-498 (1989)).

As used herein, the term transformation refers to the transfer of nucleic acid (i.e., a nucleotide polymer) into a cell. As used herein, the term genetic transformation refers to the transfer and incorporation of DNA, especially recombinant DNA, into a cell.

A construct or cassette is a package of genetic material inserted into the genome of a cell via various techniques. An embodiment provides the expression cassette comprises a nucleic acid molecule having at least a regulatory region operably linked to a nucleic acid molecule. With the present methods the cassette in an embodiment provides the YAO regulatory region operably linked to a nucleic acid molecule encoding a nuclease such as Cas9.

As used herein, the term vector refers broadly to any plasmid or virus encoding an exogenous nucleic acid. The term should also be construed to include non-plasmid and non-viral compounds which facilitate transfer of nucleic acid into virions or cells, such as, for example, polylysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, a recombinant vaccinia virus, a recombinant adenovirus, a recombinant retrovirus, a recombinant adeno-associated virus, a recombinant avian pox virus, and the like (Cranage et al., 1986, EMBO J. 5:3057-3063; U.S. Pat. No. 5,591,439). Examples of non-viral vectors include, but are not limited to, liposomes, polyamine derivatives of DNA, and the like.

Unless otherwise indicated, a particular nucleic acid sequence also implicitly encompasses conservatively modified variants thereof (e.g. degenerate codon substitutions) and complementary sequences as well as the sequence explicitly indicated. The term conservatively modified variants applies to both amino acid and nucleic acid sequences. With respect to particular nucleic acid sequences, conservatively modified variants refers to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are silent variations and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide is implicit in each described polypeptide sequence and is within the scope of the products and processes described.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” referred to herein as a “variant” where the alteration results in the substitution of an amino acid with a chemically similar amino acid. Conservative substitution tables providing functionally similar amino acids are well known in the art. See, for example, Davis et al., “Basic Methods in Molecular Biology” Appleton & Lange, Norwalk, Conn. (1994). Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., see, e.g., Creighton, Proteins: Structures and Molecular Properties (WH Freeman & Co.; 2nd edition (December 1993)).

By encoding or encoded, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the universal genetic code. However, variants of the universal code, such as are present in some plant, animal, and fungal mitochondria, the bacterium Mycoplasma capricolum, or the ciliate Macronucleus, may be used when the nucleic acid is expressed therein.

With reference to nucleic acid molecules, the term isolated nucleic acid is sometimes used. This term, when applied to DNA, refers to a DNA molecule that is separated from sequences with which it is immediately contiguous (in the 5′ and 3′ directions) in the naturally occurring genome of the organism from which it was derived. For example, the isolated nucleic acid may comprise a DNA molecule inserted into a vector, such as a plasmid or virus vector, or integrated into the genomic DNA of a prokaryote or eukaryote. An isolated nucleic acid molecule may also comprise a cDNA molecule.

When referring to hybridization techniques, all or part of a known nucleotide sequence can be used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the DNA sequences of the invention. Methods for preparation of probes for hybridization and for construction of cDNA and genomic libraries are generally known in the art and are disclosed (Sambrook et al., 2001).

For example, the sequence disclosed herein, or one or more portions thereof, may be used as a probe capable of specifically hybridizing to corresponding sequences. To achieve specific hybridization under a variety of conditions, such probes include sequences that are unique among the sequences to be screened and are preferably at least about 10 nucleotides in length, and most preferably at least about 20 nucleotides in length. Such sequences may alternatively be used to amplify corresponding sequences from a chosen plant by PCR. This technique may be used to isolate sequences from a desired plant or as a diagnostic assay to determine the presence of sequences in a plant. Hybridization techniques include hybridization screening of DNA libraries plated as either plaques or colonies (Sambrook et al., 2001).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 50° C. Exemplary high stringency conditions include hybridization in 50% formamide, 1 M NaCl, 0.1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of the complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with 90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (2001) Molecular Cloning: A Laboratory Manual (3^{rd} ed., Cold Spring Harbor Laboratory Press, Plainview, N.Y.) and Haymes et al. (1985) In: Nucleic Acid Hybridization, a Practical Approach, IRL Press, Washington, D.C.

The following terms are used to describe the sequence relationships between two or more nucleic acids or polynucleotides: (a) “reference sequence”, (b) “comparison window”, (c) “sequence identity” and (d) “percentage of sequence identity.”

In general, sequences that correspond to the nucleotide sequences described and hybridize to the nucleotide sequence disclosed herein will be at least 50% homologous, 70% homologous, and even 85%, 86%, 87%, 88%, 89%, 90%, 91%, 92%, 93%, 94%, 95%, 96%, 97%, 98%, 99% homologous or more with the disclosed sequence. That is, the sequence similarity between probe and target may range, sharing at least about 50%, about 70%, and even about 85% or more sequence similarity.

The following terms are used to describe the sequence relationships between two or more nucleic acids or polynucleotides: (a) “reference sequence”, (b) “comparison window”, (c) “sequence identity” and (d) “percentage of sequence identity.”

(a) As used herein, “reference sequence” is a defined sequence used as a basis for sequence comparison. A reference sequence may be a subset or the entirety of a specified sequence; for example, as a segment of a full-length promoter sequence, or the complete promoter sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to accurately reflect the similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm.

Optimal alignment of sequences for comparison can use any means to analyze sequence identity (homology) known in the art, e.g., by the progressive alignment method of termed “PILEUP” (Morrison, (1997) Mol. Biol. Evol. 14:428-441, as an example of the use of PILEUP); by the local homology algorithm of Smith & Waterman (Adv. Appl. Math. 2: 482 (1981)); by the homology alignment algorithm of Needleman & Wunsch (J. Mol. Biol. 48:443-453 (1970)); by the search for similarity method of Pearson (Proc. Natl. Acad. Sci. USA 85: 2444 (1988)); by computerized implementations of these algorithms (e.g., GAP, BEST FIT, FASTA, and TFASTA in the Wisconsin Genetics Software Package, Genetics Computer Group, 575 Science Dr., Madison, Wis.); ClustalW (CLUSTAL in the PC/Gene program by Intelligenetics, Mountain View, Calif., described by, e.g., Higgins (1988), Gene 73: 237-244; Corpet (1988), Nucleic Acids Res. 16:10881-10890; Huang, Computer Applications in the Biosciences 8:155-165 (1992); and Pearson (1994), Methods in Mol. Biol. 24:307-331); Pfam (Sonnhammer (1998), Nucleic Acids Res. 26:322-325); TreeAlign (Hein (1994), Methods Mol. Biol. 25:349-364); MEG-ALIGN, and SAM sequence alignment computer programs; or, by manual visual inspection.

Another example of algorithm that is suitable for determining sequence similarity is the BLAST algorithm, which is described in Altschul et al, (1990)J. Mol. Biol. 215: 403-410. The BLAST programs (Basic Local Alignment Search Tool) of Altschul, S. F., et al., searches under default parameters for identity to sequences contained in the BLAST “GENEMBL” database. A sequence can be analyzed for identity to all publicly available DNA sequences contained in the GENEMBL database using the BLASTN algorithm under the default parameters.

Software for performing BLAST analyses is publicly available through the National Center for Biotechnology Information, www.ncbi.nlm.nih.gov/; see also Zhang (1997), Genome Res. 7:649-656 for the “PowerBLAST” variation. This algorithm involves first identifying high scoring sequence pairs (HSPs) by identifying short words of length W in the query sequence that either match or satisfy some positive valued threshold score T when aligned with a word of the same length in a database sequence. T is referred to as the neighborhood word score threshold (Altschul et al (1990), J. Mol. Biol. 215: 403-410). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a wordlength (W) of 11, the BLOSUM62 scoring matrix (see Henikoff (1992), Proc. Natl. Acad. Sci. USA 89:10915-10919) alignments (B) of 50, expectation (E) of 10, M=5, N=−4, and a comparison of both strands. The term BLAST refers to the BLAST algorithm which performs a statistical analysis of the similarity between two sequences; see, e.g., Karlin (1993), Proc. Natl. Acad. Sci. USA 90:5873-5787. One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

In an embodiment, GAP (Global Alignment Program) can be used. GAP uses the algorithm of Needleman and Wunsch (J. Mol. Biol. 48:443-453, 1970) to find the alignment of two complete sequences that maximizes the number of matches and minimizes the number of gaps. Default gap creation penalty values and gap extension penalty values in the commonly used Version 10 of the Wisconsin Package® (Accelrys, Inc., San Diego, Calif.) for protein sequences are 8 and 2, respectively. For nucleotide sequences the default gap creation penalty is 50 while the default gap extension penalty is 3. Percent Similarity is the percent of the symbols that are similar. Symbols that are across from gaps are ignored. A similarity is scored when the scoring matrix value for a pair of symbols is greater than or equal to 0.50, the similarity threshold. A general purpose scoring system is the BLOSUM62 matrix (Henikoff and Henikoff (1993), Proteins 17: 49-61), which is currently the default choice for BLAST programs. BLOSUM62 uses a combination of three matrices to cover all contingencies. Altschul, J. Mol. Biol. 36: 290-300 (1993), herein incorporated by reference in its entirety and is the scoring matrix used in Version 10 of the Wisconsin Package® (Accelrys, Inc., San Diego, Calif.) (see Henikoff & Henikoff (1989) Proc. Natl. Acad. Sci. USA 89:10915).

(c) As used herein, “sequence identity” or “identity” in the context of two nucleic acid sequences makes reference to the residues in the two sequences that are the same when aligned for maximum correspondence over a specified comparison window.

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison, and multiplying the result by 100 to yield the percentage of sequence identity.

Identity to the sequence of the described here would mean a polynucleotide sequence having at least 65% sequence identity, more preferably at least 70% sequence identity, more preferably at least 75% sequence identity, more preferably at least 80% identity, more preferably at least 85% 86%, 87%, 88%, 89%, 90%, 91%, 92%, 93%, 94%, 95%, 96%, 97%, 98%, 99% sequence identity.

The sequences used here apply further to “functional variants” of the regulatory sequence disclosed. Functional variants include, for example, regulatory sequences of the invention having one or more nucleotide substitutions, deletions or insertions and wherein the variant retains promoter activity, particularly the ability to drive expression as described herein. Functional variants can be created by any of a number of methods available to one skilled in the art, such as by site-directed mutagenesis, induced mutation, identified as allelic variants, cleaving through use of restriction enzymes, or the like. Activity can likewise be measured by any variety of techniques, including measurement of reporter activity as is described at U.S. Pat. No. 6,844,484, Northern blot analysis, or similar techniques. The '484 patent describes the identification of functional variants of different promoters, incorporated herein by reference in its entirety.

By “promoter” is meant a regulatory element of DNA capable of regulating the transcription of a sequence linked thereto. It usually comprises a TATA box capable of directing RNA polymerase II to initiate RNA synthesis at the appropriate transcription initiation site for a particular coding sequence. The promoter is the minimal sequence sufficient to direct transcription in a desired manner. The term “regulatory element” in this context is also used to refer to the sequence capable of “regulatory element activity,” that is, regulating transcription in a desired manner. Therefore the invention is directed to the regulatory element described herein including those sequences which hybridize to same and have identity to same, as indicated, and fragments and variants of same which have regulatory activity.

The YAO promoter useful herein extends to functional homologs/orthologs of the promoter with mutations in corresponding/equivalent positions when compared to the YAO sequence. A functional variant or homolog is a YAO promoter which is biologically active in the same way as SEQ ID NO: 2, in other words, for example it confers increased gene editing when used in a CRISPR/Cas9 process and when compared to use of the 35S promoter. The term functional homolog includes YAO orthologs in other plant species.

Such promoters may be isolated from other plant species, using the processes described herein. By way of example, without limitation, the promoter may be obtained using these processes, whether by using the Arabidopsis or other known YAO gene, protein or promoter to identify a YAO gene, protein or promoter from another species, and where a promoter region of an identified nucleic acid molecule is identified, obtaining the promoter. Examples, without intending to be limiting, of such other plant species in addition to Arabidopsis are corn (Zea mays), millet (Setaria italic), rice (Oryza sativa), sorghum (Sorghum bicolor, Sorghum vulgare), wheat (Triticum aestivum), soybean (Glycine max), tobacco (Nicotiana tabacum), tomato (Solanum lycopersicum), potato (Solanum tuberosum), and cotton (Gossypium raimondii).

The promoter that may be used here further encompasses a “functional fragment” that is a regulatory fragment formed by one or more deletions from a larger regulatory element. For example, the 5′ portion of a promoter up to the TATA box near the transcription start site can be deleted without abolishing promoter activity, as described by Opsahl-Sorteberg, H-G. et al., 2004 Gene 341:49-58. Such fragments should retain promoter activity, particularly the ability to drive expression of operably linked nucleotide sequences. Activity can be measured by Northern blot analysis, reporter activity measurements when using transcriptional fusions, and the like. See for example, Sambrook et al. (2001). Functional fragments can be obtained by use of restriction enzymes to cleave the naturally occurring regulatory element nucleotide sequences disclosed herein; by synthesizing a nucleotide sequence from the naturally occurring DNA sequence; or can be obtained through the use of PCR technology. See particularly, Mullis et al. (1987) Methods Enzymol. 155:335-350) and Erlich, ed. (1989) PCR Technology (Stockton Press, New York).

Smaller fragments may yet contain the regulatory properties of the promoter so identified and deletion analysis is one method of identifying essential regions. Deletion analysis can occur from both the 5′ and 3′ ends of the regulatory region. Fragments can be obtained by site-directed mutagenesis, mutagenesis using the polymerase chain reaction and the like. (See, Directed Mutagenesis: A Practical Approach IRL Press (1991)). The 3′ deletions can delineate the essential region and identify the 3′ end so that this region may then be operably linked to a core promoter of choice. Once the essential region is identified, transcription of an exogenous gene may be controlled by the essential region plus a core promoter. By core promoter is meant the sequence called the TATA box which is common to promoters in all genes encoding proteins. Thus the upstream promoter of YAO can optionally be used in conjunction with its own or core promoters from other sources. The promoter may be native or non-native to the cell in which it is found.

For example, a routine way to remove a part of a DNA sequence is to use an exonuclease in combination with DNA amplification to produce unidirectional nested deletions of double stranded DNA clones. A commercial kit for this purpose is sold under the trade name Exo-Size™ (New England Biolabs, Beverly, Mass.). Briefly, this procedure entails incubating exonuclease III with DNA to progressively remove nucleotides in the 3′ to 5′ direction at the 5′ overhangs, blunt ends or nicks in the DNA template. However, the exonuclease III is unable to remove nucleotides at 3′ 4-base overhangs. Timed digest of a clone with this enzyme produces unidirectional nested deletions.

As used herein, the term “cis-element” refers to a cis-acting transcriptional regulatory element that confers an aspect of the overall control of gene expression. A cis-element may function to bind transcription factors, trans-acting protein factors that regulate transcription. Some cis-elements bind more than one transcription factor, and transcription factors may interact with different affinities with more than one cis-element. The promoters herein desirably contain cis-elements that can confer or modulate gene expression. Cis-elements can be identified by a number of techniques, including deletion analysis, i.e., deleting one or more nucleotides from the 5′ end or internal to a promoter; DNA binding protein analysis using DNase I footprinting, methylation interference, electrophoresis mobility-shift assays, in vivo genomic footprinting by ligation-mediated PCR, and other conventional assays; or by DNA sequence similarity analysis with known cis-element motifs by conventional DNA sequence comparison methods. The fine structure of a cis-element can be further studied by mutagenesis (or substitution) of one or more nucleotides or by other conventional methods. Cis-elements can be obtained by chemical synthesis or by isolation from promoters that include such elements, and they can be synthesized with additional flanking nucleotides that contain useful restriction enzyme sites to facilitate subsequent manipulation.

The YAO promoter described herein is useful in increasing gene editing frequency when used in a CRISPR/Cas9 gene editing process. This process has been explored for precise editing of a genome. See Zhang et al. U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, and Doudna et al. US Publication No. 20140068797, incorporated herein by reference in their entirety.

The YAO promoter has been found to result in exceptional increases in frequency of gene editing using the precise targeting process of Clustered, Regularly Interspaced Short Palindromic Repeats (CRISPR) which is combined with the Cas9 nuclease to make a double stranded break, the combination of which is referred to as CRISPR/Cas9 or CRISPR/Cas9 system. The site of the break is targeted by short guide RNA often about 20 nucleotides. The break can be repaired by non-homologous end joining (NHEJ) or homology-directed recombination. CRISPR is an adaptive immune system that provides protection against mobile genetic elements (viruses, transposable elements and conjugative plasmids) first discovered in bacteria. CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA uses a trans-encoded small RNA (tracrRNA), endogenous ribonuclease 3 (rnc) and a Cas9 protein. The term Cas9 or “Cas9 nuclease” refers to an RNA-guided nuclease comprising a Cas9 protein, or a fragment thereof (e.g., a protein comprising an active or inactive DNA cleavage domain of Cas9, and/or the gRNA binding domain of Cas9). A Cas9 nuclease is also referred to sometimes as a casn1 nuclease or a CRISPR associated nuclease. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNA. However, single guide RNAs (“sgRNA”, or simply “gNRA”) can be engineered so as to incorporate aspects of both the crRNA and tracrRNA into a single RNA species. See e.g., Jinek et al. Science 337:816-821 (2012). Cas9 recognizes a short motif in the CRISPR repeat sequences (the PAM or protospacer adjacent motif) to help distinguish self versus non-self. Cas9 nuclease sequences and structures are well known to those of skill in the art (see, e.g., Ferretti et al “Complete genome sequence of an Ml strain of Streptococcus pyogenes, Proc. Natl. Acad. Sci. U.S.A. 98:4658-4663 (2001); Deltcheva et al. “CRISPR RNA maturation by trans-encoded small RNA and host factor RNase III”, Nature 471:602-607 (2011); and Jinek et al. “A programmable dual-RNA-guided DNA endonuclease in adaptive bacterial immunity.” Science 337:816-821 (2012)). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include, for example, Cas9 sequences from the organisms and loci disclosed in Chylinski et al., “The tracrRNA and Cas9 families of type II CRISPR-Cas immunity systems” (2013) RNA Biology 10:5, 726-737. In some embodiments, a Cas9 nuclease has an inactive (e.g., an inactivated) DNA cleavage domain. A nuclease-inactivated Cas9 protein may interchangeably be referred to as a “dCas9” protein (for nuclease “dead” Cas9). By way of example of the many variants available to one skilled in the art, see Liu et al. U.S. Pat. No. 9,388,430, incorporated herein by reference in its entirety.

The promoter in an embodiment is useful with Transcription Activator-Like Effector Nucleases or TALENs. These transcription factor nucleases are useful in precise gene editing and have domains with repeats of amino acids capable of recognizing a base pair in a DNA sequence. There is a hypervariable region of two residues, and this determines DNA binding specificity. See for example Bonas et al. U.S. Pat. No. 8,420,782, Voytas et al. U.S. Pat. Nos. 8,440,431, 8,440,432, and 8,697,853, incorporated by reference herein in their entirety. The specific embodiment of the TALEN process may vary depending upon the goal of the alteration and advances in development of the process. In one example, without intending to be limiting, the hybervariable region which determines recognition of a base pair can, in one example be selected from: (a) HD for recognition of C/G; (b) NI for recognition of A/T; (c) NG for recognition of T/A; (d) NS for recognition of C/G or A/T or T/A or G/C; (e) NN for recognition of G/C or A/T; (f) IG for recognition of T/A; (g) N for recognition of C/G; (h) HG for recognition of C/G or T/A; (i) H for recognition of T/A; and (j) NK for recognition of G/C. Still other variations exist and the process here is not limited to this example. The TAL effector domain that binds to a specific nucleotide sequence within the target DNA can in one embodiment comprise 10 or more DNA binding repeats, and preferably 15 or more DNA binding repeats. Each DNA binding repeat can include a repeat variable-diresidue (RVD) that determines recognition of a base pair in the target DNA sequence, wherein each DNA binding repeat is responsible for recognizing one base pair in the target DNA sequence

Breaking DNA using site specific endonucleases can increase the rate of homologous recombination in the region of the breakage. In some embodiments, the FokI (Flavobacterium okeanokoites) endonuclease may be utilized in an effector to induce DNA breaks. The Fok I endonuclease domain functions independently of the DNA binding domain and cuts a double stranded DNA typically as a dimer (Li et al. (1992) Proc. Natl. Acad. Sci. U.S.A 89 (10):4275-4279, and Kim et al. (1996) Proc. Natl. Acad. Sci. U.S.A 93 (3):1156-1160). A single-chain FokI dimer has also been developed and could also be utilized (Mino et al. (2009) J. Biotechnol. 140:156-161). An effector could be constructed that contains a repeat domain for recognition of a desired target DNA sequence as well as a FokI endonuclease domain to induce DNA breakage at or near the target DNA sequence similar to previous work done employing zinc finger nucleases (Townsend et al. (2009) Nature 459:442-445; Shukla et al. (2009) Nature 459, 437-441). Utilization of such effectors could enable the generation of targeted changes in genomes which include additions, deletions and other modifications, analogous to those uses reported for zinc finger nucleases as per Bibikova et al. (2003) Science 300, 764; Urnov et al. (2005) Nature 435, 646; Wright et al. (2005) The Plant Journal 44:693-705; and U.S. Pat. Nos. 7,163,824 and 7,001,768, incorporated by reference in their entireties. An example of a method to modulate the expression of a target gene in plant cells comprises the following steps: a) providing plant cells with an expression system for a polypeptide capable of specifically recognizing, and preferably binding, to a target nucleotide sequence, or a complementary strand thereof; and b) culturing the plant cells under conditions wherein said polypeptide is produced and binds to said target nucleotide sequence, whereby expression of said target gene in said plant cells is modulated.

In one example, a method for producing a polypeptide that selectively recognizes at least one base pair in a target DNA sequence may be employed, comprising synthesizing a polypeptide comprising a repeat domain, wherein the repeat domain comprises at least one repeat unit derived from a transcription activator-like (TAL) effector, wherein the repeat unit comprises a hypervariable region which determines recognition of a base pair in the target DNA sequence, wherein the repeat unit is responsible for the recognition of one base pair in the DNA sequence. The method may utilize an expression cassette comprising a promoter operably linked to the above-mentioned DNA.

Another gene altering technology uses the transcription factors of zinc fingers, where zinc finger nucleases are heterodimers formed of a zinc finger domain and a nuclease, in an embodiment a FokI endonuclease domain. Target specificity is provided when the FokI domains dimerize to cause cleavage. The zinc finger DNA binding protein or binding domain binds DNA in a sequence specific manner through at least one zinc finger, that is, amino acid regions with structure stabilized by a zinc ion. These zinc finger proteins are designed to bind to a predetermined nucleotide sequence. Many approaches exists and examples of such designs are found at, for example, Pavletich et al. (1991) “Zinc finger-DNA recognition: crystal structure of a Zif268-CAN complex at 2.1A” Science 252 (5007): 809-17; Rebar et al. (1994) “Zinc finger phase: affinity selection of fingers with new DNA-binding specificities” Science 263 (5147): 671-3US and U.S. Pat. Nos. 6,140,081; 6,453,242; 6,534,261, the contents of which are incorporated herein by reference in their entirety. A vast array of methods are available to one skilled in the art for producing zinc finger binding domains and the methods here are not limited to a specific process. Modular assembly and use of a bacterial selections system are two such systems used. In one separate zinc fingers recognizing three base pair sequences are provided to generate arrays that can recognize longer target sites.

Any target gene (referring to an entire gene or a single nucleotide sequence) can be modulated by the present method. When referring to altering or editing a targeted nucleic acid molecule is meant to include various forms of changing the targeted gene or its expression. The process may be used to alter a target gene, that is to edit, modify or change a single nucleotide, multiple nucleotides, or for deletion of a large fragment, substitutions and insertions of sequences. The target nucleotide sequence can be present in a living cell or present in vitro. In a specific embodiment, the target nucleotide sequence is endogenous to the plant. The target nucleotide sequence can be located in any suitable place in relation to the target gene. For example, the target nucleotide sequence can be upstream or downstream of the coding region of the target gene. Alternatively, the target nucleotide sequence is within the coding region of the target gene. The target nucleotide sequence can also be a promoter of a gene. For example, the target gene can encode a product that affects biosynthesis, modification, cellular trafficking, metabolism and degradation of a peptide, a protein, an oligonucleotide, a nucleic acid, a vitamin, an oligosaccharide, a carbohydrate, a lipid, or a small molecule. Furthermore, the process can be used to engineer plants for traits such as increased disease resistance, modification of structural and storage polysaccharides, flavors, proteins, and fatty acids, fruit ripening, yield, color, nutritional characteristics, improved storage capability, and the like.

As described further herein, measuring and detecting the presence of an edited target nucleic acid molecule may use any convenient method, and will depend upon the desired editing, whether addition, deletion or other modification of the genome. Restriction fragment length polymorphism analysis, polymerase chain reaction analysis, Northern, Southern or Western blot analysis, other genotypic analysis, measurement of reporter activity or phenotype analysis are a few examples of the myriad ways in which a person skilled in the art may analyze whether the targeted nucleic acid molecule is changed after use of the processes and components described herein.

In addition, the cassette may advantageously comprise functional domains from other proteins (e.g. catalytic domains from restriction endonucleases, recombinases, replicases, integrases and the like). The polypeptide may also comprise activation or processing signals, such as nuclear localisation signals. These are of particular usefulness in targeting the polypeptide to the nucleus of the cell in order to enhance the binding of the polypeptide to an intranuclear target (such as genomic DNA). The following are examples of components that may be used in the cassettes and processes described here and are not intended to be limiting.

In one embodiment, the Cas9 nuclease can be following b1) or b2):

b1) a protein having an amino acid sequence shown by SEQ ID NO: 8; or
b2) a protein having the same function as the Cas9 nuclease, which is obtained by subjecting the protein shown by b1) to substitutions and/or deletions and/or additions of 1 to 10 amino acid residues.

The expression cassette I can include following elements in sequence from 5′ end to 3′ end: the promoter pYAO, the coding gene of the Cas9 nuclease, and a terminator. The coding gene of the Cas9 nuclease can be shown by bases 1139-5239 (SEQ ID NO: 5) from 5′ terminal end in SEQ ID NO: 1. The terminator in an embodiment is a NOS terminator. The nucleotide sequence of the NOS terminator can be shown by bases 5297-5580 (SEQ ID NO: 7) from 5′ terminal end in SEQ ID NO: 1. The expression cassette I can also include more than one Flag tags and/or more than one nuclear localization signals. The expression cassette I can in an embodiment include one Flag tag, a nuclear localization signal I and a nuclear localization signal II. The expression cassette I can include following elements in sequence from 5′ end to 3′ end: the promoter pYAO, the Flag tag, the nuclear localization signal I, the coding gene of Cas9 nuclease, the nuclear localization signal II and a terminator. The nucleotide sequence of the Flag tag can particularly be shown by bases 1019-1087 (SEQ ID NO: 3) from 5′ terminal end in SEQ ID NO: 1. The nucleotide sequence of the nuclear localization signal I can particularly be shown by bases 1088-1138 (SEQ ID NO: 4) from 5′ terminal end in SEQ ID NO: 1. The nucleotide sequence of the nuclear localization signal II can particularly be shown by bases 5240-5287 (SEQ ID NO: 6) from 5′ terminal end in SEQ ID NO: 1. The nucleotide sequence of the expression cassette I can particularly be shown by SEQ ID NO: 1. The initiation of the coding gene of Cas9 nuclease can particularly be to initiate the expression of the coding gene of Cas9 nuclease in plants.

A recombinant plasmid containing any one of above expression cassette may be used with the YAO promoter. The recombinant plasmid can also include an expression cassette II, in which sgRNA transcription is initiated by an AtU6-26 promoter. The expression cassette II can include an AtU6-26 promoter and a sgRNA segment (the sgRNA segment is a DNA fragment having the coding gene of sgRNA) in sequence from 5′ end to 3′ end. The sgRNA segment can include a crRNA segment (the crRNA segment is a fragment having the coding gene of crRN) and a tracrRNA segment (the tracrRNA segment is a fragment having the coding gene of tracrRNA).

The crRNA specifically binds to a target fragment in the target gene, the target fragment can have following structures: 5′-N_{X}-NGG-3′, N represents any one of A, G, C, and T, and X=20. The nucleotide sequence of the crRNA segment can particularly be shown by bases 9390-9409 (SEQ ID NO: 21) from 5′ terminal end in SEQ ID NO: 21. The nucleotide sequence of the tracrRNA segment in one embodiment may be the sequence of bases 9410-9485 (SEQ ID NO: 25) from 5′ terminal end in SEQ ID NO: 21. It is to be understood that in referring to expression cassette I or II is used for ease of referencing operably linked components to promoters and is not intended to require a particular vector or cassette formation or processes of producing the components. In the expression cassette II, a 3′-UTR segment can also be included downstream of the sgRNA segment. The nucleotide sequence of the 3′-UTR segment can particularly be shown by bases 9493-9575 (SEQ ID NO: 26) from 5′ terminal end in SEQ ID NO: 21. The nucleotide sequence of the expression cassette II in one example include bases 8941-9575 (SEQ ID NO: 23) from 5′ terminal end in SEQ ID NO: 21.

The recombinant plasmid can also include a functional fragment II, and the functional fragment II can include an AtU6-26 promoter, a multiple cloning site segment into which the coding gene of crRNA is to be inserted, and a tracrRNA segment in sequence from 5′ end to 3′ end.

The crRNA specifically binds to a target fragment in the target gene, the target fragment has following structures: 5′-N_{X}-NGG-3′, N represents any one of A, G, C, and T, and X=20.

The multiple cloning site segments can include more than one restriction recognition sites of restriction enzyme BsaI, and can in an embodiment have two restriction recognition sites of restriction enzyme BsaI. The nucleotide sequences of the two restriction recognition sites of restriction enzyme BsaI can be shown by bases 451-456 (SEQ ID NO: 16) and bases 465-470 (SEQ ID NO: 17) from 5′ terminal end in SEQ ID NO: 13, respectively. The nucleotide sequence of the multiple cloning site segment can particularly be shown by bases 449-471 (SEQ ID NO: 15) from 5′ terminal end in SEQ ID NO: 13. The nucleotide sequence of the AtU6-26 promoter can particularly be shown by Sites 1-448 (SEQ ID NO: 13) from 5′ terminal end in SEQ ID NO: 13. The nucleotide sequence of the tracrRNA segment can particularly be shown by bases 472-547 (SEQ ID NO: 18) from 5′ terminal end in (SEQ ID NO: 13). In the functional fragment II, a 3′-UTR segment can also be included downstream of the tracrRNA segment. The nucleotide sequence of the 3′-UTR segment can particularly be shown by bases 555-637 (SEQ ID NO: 19) from 5′ terminal end in SEQ ID NO:). The nucleotide sequence of the functional segment II can particularly be shown by SEQ ID NO: 13.

The present disclosure also provides a method for directed editing of plant genomes.

By way of example, a method for directed editing of plant genomes provided by the present invention is Method (c1) or Method (c2):

Method (c1) may include a following step: directly editing the target gene of the sgRNA in the genome of an original plant by introducing a recombinant plasmid containing any one of above expression cassette IIs into the original plant.
Method (c2) includes following steps: (1) designing crRNA according to the target gene anticipated to be directedly edited in the original plant; (2) inserting the coding gene of the crRNA into the multiple cloning site segment of the recombinant plasmid containing any one of the above functional segment IIs, to obtain a recombinant plasmid I; and (3) introducing the recombinant plasmid I into the original plant, thereby directly editing the target gene in the genome of the original plant.

The system for directed editing of plant genomes provided by the present invention includes a recombinant plasmid expressing a CRISPR/Cas9 system, characterized in that: the promoter initiating the Cas9 expression in the recombinant plasmid is any one of the above promoter pYAOs.

The promoter pYAO also falls into the scope of the present disclosure. The use of the promoter pYAO for the initiation of the expression of a gene of interest also falls into the scope of the present disclosure.

The gene of interest can in an embodiment be the coding gene of a Cas9 nuclease. The Cas9 nuclease can be following b1) or b2): b1) a protein having a amino acid sequence shown by SEQ ID NO: 8; or b2) a protein having the same function as the Cas9 nuclease, which is obtained by subjecting the protein shown by b1) to substitutions and/or deletions and/or additions of 1 to 10 amino acid residues. The coding gene of the Cas9 nuclease is in one embodiment shown at bases 1139-5239 (SEQ ID NO: 5) from 5′ terminal end in SEQ ID NO: 1.

The term introduced in the context of inserting a nucleic acid into a cell, includes transfection or transformation or transduction and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA). When referring to introduction of a nucleotide sequence into a plant is meant to include transformation into the cell, as well as crossing a plant having the sequence with another plant, so that the second plant contains the heterologous sequence, as in conventional plant breeding techniques. Such breeding techniques are well known to one skilled in the art. For a discussion of plant breeding techniques, see Poehlman (1995) Breeding Field Crops. AVI Publication Co., Westport Conn., 4^{th} Edit. Backcrossing methods may be used to introduce a gene into the plants. This technique has been used for decades to introduce traits into a plant. An example of a description of this and other plant breeding methodologies that are well known can be found in references such as Poelman, supra, and Plant Breeding Methodology, edit. Neal Jensen, John Wiley & Sons, Inc. (1988). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the single transferred gene from the nonrecurrent parent.

As used herein, a nucleotide segment is referred to as operably linked when it is placed into a functional relationship with another DNA segment. For example, DNA for a signal sequence is operably linked to DNA encoding a polypeptide if it is expressed as a preprotein that participates in the secretion of the polypeptide; a promoter or enhancer is operably linked to a coding sequence if it stimulates the transcription of the sequence. Operably linked elements may be contiguous or non-contiguous. When used to refer to the joining of two protein coding regions, by operably linked it is intended that the coding regions are in the same reading frame. Alternatively, the additional gene(s) can be provided on multiple expression cassettes. Such an expression cassette is provided with a plurality of restriction sites and/or recombination sites for insertion of the polynucleotide to be under the transcriptional regulation of the regulatory regions. The expression cassette can include one or more enhancers in addition to the promoter. By enhancer is intended a cis-acting sequence that increases the utilization of a promoter. Such enhancers can be native to a gene or from a heterologous gene. Further, it is recognized that some promoters can contain one or more enhancers or enhancer-like elements. An example of one such enhancer is the 35S enhancer, which can be a single enhancer, or duplicated. See for example, McPherson et al, U.S. Pat. No. 5,322,938.

The method of transformation/transfection is not critical to the instant invention; various methods of transformation or transfection are currently available. As newer methods are available to transform crops or other host cells they may be directly applied. Accordingly, a wide variety of methods have been developed to insert a DNA sequence into the genome of a host cell to obtain the transcription or transcript and translation of the sequence to effect phenotypic changes in the organism. Thus, any method which provides for efficient transformation/transfection may be employed.

Methods for introducing expression vectors into plant tissue available to one skilled in the art are varied and will depend on the plant selected. Procedures for transforming a wide variety of plant species are well known and described throughout the literature. (See, for example, Miki and McHugh (2004) Biotechnol. 107, 193-232; Klein et al. (1992) Biotechnology (N Y) 10, 286-291; and Weising et al. (1988) Annu. Rev. Genet. 22, 421-477). For example, the DNA construct may be introduced into the genomic DNA of the plant cell using techniques such as microprojectile-mediated delivery (Klein et al. 1992, supra), electroporation (Fromm et al., 1985 Proc. Natl. Acad. Sci. USA 82, 5824-5828), polyethylene glycol (PEG) precipitation (Mathur and Koncz, 1998 Methods Mol. Biol. 82, 267-276), direct gene transfer (WO 85/01856 and EP-A-275 069), in vitro protoplast transformation (U.S. Pat. No. 4,684,611), and microinjection of plant cell protoplasts or embryogenic callus (Crossway, A. (1985) Mol. Gen. Genet. 202, 179-185). Agrobacterium transformation methods of Ishida et al. (1996) and also described in U.S. Pat. No. 5,591,616 are yet another option. Co-cultivation of plant tissue with Agrobacterium tumefaciens is a variation, where the DNA constructs are placed into a binary vector system (Ishida et al., 1996 Nat. Biotechnol. 14, 745-750). The virulence functions of the Agrobacterium tumefaciens host will direct the insertion of the construct into the plant cell DNA when the cell is infected by the bacteria. See, for example, Fraley et al. (1983) Proc. Natl. Acad. Sci. USA, 80, 4803-4807. Agrobacterium is primarily used in dicots, but monocots including maize can be transformed by Agrobacterium. See, for example, U.S. Pat. No. 5,550,318. In one of many variations on the method, Agrobacterium infection of corn can be used with heat shocking of immature embryos (Wilson et al. U.S. Pat. No. 6,420,630) or with antibiotic selection of Type II callus (Wilson et al., U.S. Pat. No. 6,919,494).

Rice transformation is described by Hiei et al. (1994) Plant J. 6, 271-282 and Lee et al. (1991) Proc. Nat. Acad. Sci. USA 88, 6389-6393. Standard methods for transformation of canola are described by Moloney et al. (1989) Plant Cell Reports 8, 238-242. Corn transformation is described by Fromm et al. (1990) Biotechnology (N Y) 8, 833-839 and Gordon-Kamm et al. (1990) supra. Wheat can be transformed by techniques similar to those used for transforming corn or rice. Sorghum transformation is described by Casas et al. (Casas et al. (1993) Transgenic sorghum plants via microprojectile bombardment. Proc. Natl. Acad. Sci. USA 90, 11212-11216) and barley transformation is described by Wan and Lemaux (Wan and Lemaux (1994) Generation of large numbers of independently transformed fertile barley plants. Plant Physiol. 104, 37-48). Soybean transformation is described in a number of publications, including U.S. Pat. No. 5,015,580.

It is shown here that, plant genomes can be high efficiently edited by utilizing promoters of genes highly expressed during plant gametophytes or/and early embryo development, such as the promoter of YAO gene, to initiate the expression of the coding gene of the Cas9 nuclease.

The present disclosure is further described in detail below along with detailed embodiments, and the examples are given only for illustrating the present invention, not for limiting the scope of the present invention. All references cited herein are incorporated herein by reference in their entirety.

### EXAMPLES

The experimental methods in below examples, without otherwise specified, are all conventional methods. The materials, reagents etc. used in below examples, without otherwise specified, are all commercially available.

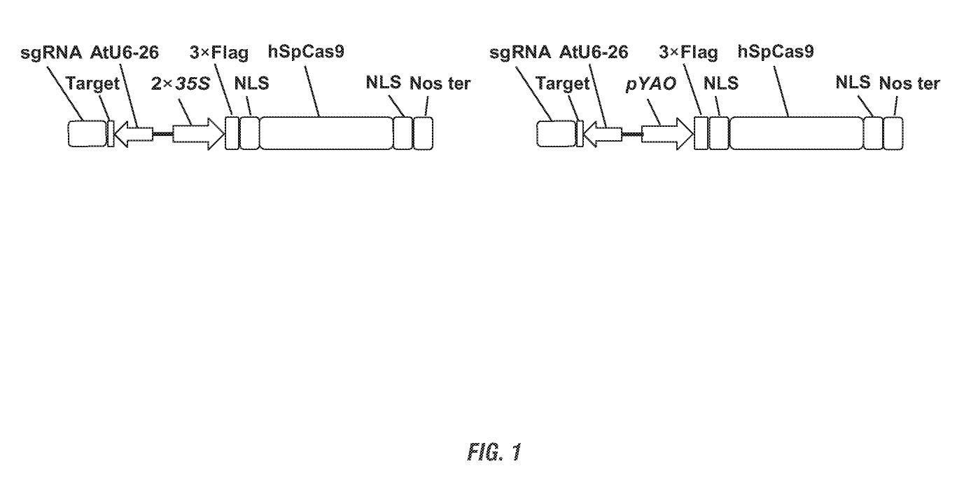

The 35S promoter and the YAO promoter were used in two binary vectors driving the same sequence encoding Cas9. Two isocaudomer restriction enzymes, SpeI and NheI were used for the left and right borders of a cassette, AtU6-26-target sgRNA providing for multiplex target sites to be assembled into the same construct. Following digestion of the vectors by the enzymes, they were inserted into the Spe I site in the 35S:hpCas9 and pYAO:hpCas9 constructs to provide a CRISPR/Cas9 system. See FIG. 1.

The wild-type Arabidopsis thaliana (Columbia-0 ecotype) is readily available (Kim H, Hyun Y, Park J, Park M, Kim M, Kim H, Lee M, Moon J, Lee I, Kim J. A genetic link between cold responses and flowering time through FVE in Arabidopsis thaliana. Nature Genetics. 2004, 36: 167-171) used in following examples from Institute of Genetics and Developmental Biology, Chinese Academy of Sciences, so as to repeat the experiments of the present application. Arabidopsis thaliana (Columbia-0 ecotype) hereinafter is referred to as wild-type Arabidopsis thaliana for short.

The vector 35S-Cas9-SK in following examples is recorded in the following literature: Feng et al. Efficient genome editing in plants using a CRISPR/Cas system. Cell Res. 2013., which can be obtained by the public from Shanghai Center for Plant Stress Biology, Chinese Academy of Sciences, so as to repeat the experiments of the present application. The vector pCAMBIA1300 and vector pBluescript-SK(+) are both products of Biovector Corporation, and KOD-Plus-Neo is a product of TOYOBO Corporation. The Arabidopsis gene BRASSINOSTEROID INSENSITIVE 1 (BRI1) was selected to show loss of function plants with a resulting dwarf phenotype. The bri1 mutant in following examples is recorded in the following literature: Noguchi, T., Fujioka, S., et al. Brassinosteroid-insensitive dwarf mutants of Arabidopsis accumulate brassinosteroids. Plant Physiol. 1999. 121:743-752. The phenotype of bri1 mutant is stunted plant, contorted lamina, prolonged vegetative growth cycle, and changed skotomorphogenesis etc.

### Example 1, Construction of a Recombinant Plasmid

1. Construction of the Recombinant Plasmid pYAO:Cas9
1) A double-stranded DNA molecule containing restriction enzyme SalI at both N end and C end was obtained by the PCR amplification with KOD-Plus-Neo using genome DNA of wild-type Arabidopsis thaliana as a template, and artificially synthesized pYAO-F: 5′-AAGTCGACGATGGGAAATTCATTGAAAACCCT-3′ (SEQ ID NO: 27) (underline portion is the SalI enzyme cleavage site) and pYAO-R: 5′-AAGTCGACTCCTTTCTTCTTCTCGTTGTTGT-3′ (SEQ ID NO: 28) (underline portion is the SalI enzyme cleavage site) as primers.
2) After step 1) was completed, single enzyme cleavage of the double-stranded DNA molecule obtained via amplification in step 1) was performed with a restriction enzyme SalI, and Fragment 1 of about 1022 bp was recovered.
3) Single enzyme cleavage of vector 35S-Cas9-SK was performed with a restriction enzyme XhoI, and Vector Backbone 1 of about 7493 bp was recovered.
4) Fragment 1 was linked with Vector Backbone 1, to obtain the recombinant plasmid pYAO-Cas9-SK.
5) Double enzyme cleavage of vector pCAMBIA1300 was performed with restriction enzymes XbaI and KpnI, and Vector Backbone 2 of about 8948 bp was recovered.
6) The artificially synthesized single-stranded DNA molecule MCS-F: 5′-CTAGATCACTAGTATCCTAGGAAGGTAC-3′ (SEQ ID NO: 29) (underline portion is the restriction recognition site of restriction enzyme SpeI, double underline portion is the sticky end of restriction enzyme XbaI, and wavy line portion is the sticky end of restriction enzyme KpnI) and single-stranded DNA molecule MCS-R: 5′-CTTCCTAGGATACTAGTGAT-3′ (SEQ ID NO: 30) (underline portion is the restriction recognition site of restriction enzyme SpeI) were mixed in a molar ratio of 1:1, and then annealed (annealing procedure comprised: 95° C. for 5 min, naturally cooling to room temperature), to form a double-stranded DNA molecule, which was named Fragment 2.
7) Vector Backbone 2 was linked with Fragment 2, to obtain the recombinant plasmid pCAMBIA1300-SpeI.
8) Double enzyme cleavage of the plasmid pCAMBIA1300-SpeI obtained in step 7) was performed with restriction enzymes KpnI and EcoRI, and Vector Backbone 3 of about 8956 bp was recovered.
9) Double enzyme cleavage of the recombinant plasmid pYAO-Cas9-SK obtained in step 4) was performed with restriction enzymes KpnI and EcoRI, and Fragment 3 of about 5597 bp was recovered.
10) Vector Backbone 3 was linked with Fragment 3, to obtain the recombinant plasmid pYAO:Cas9.
The recombinant plasmid pYAO:Cas9 expresses the Cas9 nuclease shown by (SEQ ID NO: 8).

The recombinant plasmid pYAO:Cas9 was subjected to enzyme cleavage identification and sequencing, the recombinant plasmid pYAO:Cas9 has one expression cassette I, the nucleotide sequence of which is like the DNA molecule shown by Sequence 1, wherein Sites 1-1012 (SEQ ID NO: 2) from 5′ terminal end in Sequence 1 (SEQ ID NO: 1) is pYAO promoter, Sites 1019-1087 (SEQ ID NO: 3) is a Flag tag, Sites 1088-1138 (SEQ ID NO: 4) is a nuclear localization signal, Sites 1139-5239 (SEQ ID NO: 5) is the coding gene of Cas9 nuclease, Sites 5240-5287 (SEQ ID NO: 6) is a nuclear localization signal, and Sites 5297-5580 is a NOS terminator (SEQ ID NO: 7).

### 2. Construction of Recombinant Plasmid AtU6-26-sgRNA-SK

1) A point mutation on the Bsa I enzyme cleavage site in Ampr coding region within vector pBluescript-SK(+) was performed without affecting amino acids encoded by genes, the vector subjected to the point mutation was named vector pBluescript-SK(+)-M. The construction process of vector pBluescript-SK(+)-M was as follows:

(a) The PCR amplification products were obtained by PCR amplification with KOD-Plus-Neo using vector pBluescript-SK(+) as a template, and artificially synthesized Amp^{r}BsaI-mutant F: 5′-GGCCCCAGTGCTGCAATGATACCGCGCGACCCACGCTCAC-3′ (SEQ ID NO: 31) (underline portion is the point mutation site) and Amp^{r}BsaI-mutant R: 5′-GTGAGCGTGGGTCGCGCGGTATCATTGCAGCACTGGGGCC-3′ (SEQ ID NO: 32) (underline portion is the point mutation site) as primers. PCR amplification procedure comprised: 95° C. for 5 min; 95° C. for 30 s, 55° C. for 30 s, 68° C. for 2 min, 20 cycles: and 68° C. for 10 min.

(b) Enzyme cleavage (37° C. for 30 min) of the PCR amplification products obtained in step (a) was performed with Dpn I (a product of NEB Corporation), to obtain the enzyme cleaved products. The purpose of this step was to digest the vector pBluescript-SK(+) added into the PCR system, that is, to remove the vector pBluescript-SK(+) where BsaI in Amp^{r} coding region was not mutated.

(c) After step (b) was completed, 1 μL enzyme cleaved products was taken to transform E. coli DH5α, monoclone picked, plasmid extracted for sequencing, and the recombinant plasmid pBluescript-SK(+)-M was obtained. The difference between recombinant plasmid pBluescript-SK(+)-M and plasmid pBluescript-SK(+) only lies in that the former contains the mutation sites shown in Amp^{r}BsaI-mutant F and Amp^{r}BsaI-mutant R sequences.

2) Enzyme cleavage sites of NheI were introduced into vector pBluescript-SK(+)-M, and specific steps were as follows:

(a) The PCR amplification products were obtained by the PCR amplification with KOD-Plus-Neo using the vector pBluescript-SK(+)-M constructed in step 1) as a template, and artificially synthesized CS-F: 5′-CACTATAGGGCGAATTGGGTGCTAGCCCCCCCCTCGAGGTCGAC-3′ (SEQ ID NO: 33) (underline portion is the restriction recognition site of restriction enzyme NheI, and double underline portion is the restriction recognition site of restriction enzyme XhoI) and CS-R: 5′-GTCGACCTCGAGGGGGGGGCTAGCACCCAATTCGCCCTATAGTG-3′ (SEQ ID NO: 34) (underline portion is the restriction recognition site of restriction enzyme NheI, and double underline portion is the restriction recognition site of restriction enzyme XhoI) as primers. PCR amplification procedure comprised: 95° C. for 5 min; 95° C. for 30 s, 55° C. for 30 s, 68° C. for 2 min, 20 cycles: and 68° C. for 10 min.

(b) Enzyme cleavage (37° C. for 30 min) of the PCR amplification products obtained in step (a) was performed with DpnI (a product of NEB Corporation), to obtain the enzyme cleaved products.

(c) After step (b) was completed, 1 μL, enzyme cleaved products was taken to transform E. coli DH5α, monoclone picked, plasmid extracted for sequencing, and the recombinant plasmid pBluescript-SK(+)-NheI was obtained. The difference between recombinant plasmid pBluescript-SK(+)-NheI and plasmid pBluescript-SK(+)-M only lies in that the former contains the NheI restriction recognition sites shown in CS-F and CS-R sequences.

3) The double-stranded DNA molecule containing restriction enzyme NheI at N end and restriction enzyme EcoRI at C end was obtained by the PCR amplification with KOD-Plus-Neo (a product of TOYOBO Corporation) using genome DNA of wild-type Arabidopsis thaliana as a template, and artificially synthesized AtU6-26-F: 5′-AAGCTAGCAAGCTTCGTTGAACAACGGAAACTC-3′ (SEQ ID NO: 35) (underline portion is the restriction recognition site of NheI enzyme) and AtU6-26-R: 5′-AAGAATTCAGGTCTCACAATCACTACTTCGACTCTAGCTGT-3′ (SEQ ID NO: 36) (underline portion is the restriction recognition site of EcoRI enzyme) as primers. 4) After step 3) was completed, double enzyme cleavage of the double-stranded DNA molecule obtained via amplification in step 3) was performed with restriction enzymes NheI and EcoRI, and Fragment 4 of 454 bp was recovered.
5) Double enzyme cleavage of recombinant plasmid pBluescript-SK(+)-NheI obtained in step 2) was performed with restriction enzymes NheI and EcoRI, and Vector Backbone 4 of about 2913 bp was recovered.
6) Vector Backbone 4 was linked with Fragment 4, to obtain the recombinant plasmid pBluescript-SK(+)-AtU6-26.
7) Double enzyme cleavage of vector pBluescript-SK(+)-AtU6-26 was performed with restriction enzymes EcoRI and SpeI, and Vector Backbone 5 of about 3406 bp was recovered.
8) The artificially synthesized single-stranded DNA molecule sgRNA-F and single-stranded DNA molecule sgRNA-R were mixed in a molar ratio of 1:1, and annealed (annealing procedure comprised: 95° C. for 5 min, naturally cooling to room temperature), to form the a double-stranded DNA molecule having sticky ends, which was named Fragment 5. The nucleotide sequence of sgRNA-F is like the single-stranded DNA molecule shown by (SEQ ID NO: 9), and the nucleotide sequence of sgRNA-R is like the single-stranded DNA molecule shown by (SEQ ID NO: 10).
9) The artificially synthesized single-stranded DNA molecule 3′-UTR-F and single-stranded DNA molecule 3′-UTR-R were mixed in a molar ratio of 1:1, and then annealed (annealing procedure comprised: 95° C. for 5 min, naturally cooling to room temperature), to form a double-stranded DNA molecule having sticky ends, which was named Fragment 6. The nucleotide sequence of 3′-UTR-F is like the single-stranded DNA molecule shown by (SEQ ID NO: 11), and the nucleotide sequence of 3′-UTR-R is like the single-stranded DNA molecule shown by (SEQ ID NO: 12).
10) Vector Backbone 5, Fragment 5 and Fragment 6 (the molar mass ratio of Fragment 5 to Fragment 6 is 1:1) were mixed for linking, to obtain the recombinant plasmid AtU6-26-sgRNA-SK.

The recombinant plasmid AtU6-26-sgRNA-SK was subjected to enzyme cleavage identification and sequencing, and the recombinant plasmid AtU6-26-sgRNA-SK has one functional segment II, the nucleotide sequence of which is like the double-stranded DNA molecule shown by SEQ ID NO: 13, wherein bases 1-448 (SEQ ID NO: 14) from 5′ terminal end in SEQ ID NO: 13 is AtU6-26 promoter, bases 451-456 (SEQ ID NO: 16) and Sites 465-470 (SEQ ID NO: 17) are both enzyme cleavage sites (for insertion of coding sequence of crRNA) of restriction enzyme BsaI, bases 472-547 (SEQ ID NO: 18) is the nucleotide sequence of tracrRNA segment, and bases 555-637 (SEQ ID NO: 19) is the nucleotide sequence of 3′-UTR segment.

### Example 2, Site-Directed Editing of Endogenous Gene BRI1 of Arabidopsis thaliana by pYAO:Cas9/AtU6-26-sgRNA System

### I). Design of Target Fragment BRI1-T1

The target fragment BRI1-T1 was designed, wherein the target fragment BRI1-T1 is located in the gene of interest, and one strand of double-stranded target fragment has following structures: 5′-N_{X}-NGG-3′, N represents any one of A, G, C, and T, and X=20.

The nucleotide sequence of target fragment BRI1-T1 is: 5′-TTGGGTCATAACGATATCTC-3′ (SEQ ID NO: 37) (underline portion is the restriction recognition site of EcoR V).

II). Construction of Recombinant Plasmid pYAO: hspCas9-BRI1-sgRNA
(1) BRI1-T1 F: 5′-ATTGTTGGGTCATAACGATATCTC-3′ (SEQ ID NO: 38) (underline portion is the sticky end) and BRI1-T1 R: 5′-AAACGAGATATCGTTATGACCCAA-3′ (SEQ ID NO: 39) (underline portion is the sticky end) were artificially synthesized, and BRI1-T1 F and BRI1T1 R are both single-stranded DNA molecules.
(2) BRI1-T1 F and BRI1-T1 R were mixed in a molar ratio of 1:1, and annealed (annealing procedure comprised: 95° C. for 5 min, naturally cooling to room temperature), to obtain a double-stranded DNA molecule having sticky ends.
(3) The recombinant plasmid AtU6-26-sgRNA-SK was enzymatically cleaved with BsaI enzyme (a product of NEB Corporation), then linked with the double-stranded DNA synthesized in step
(2), wherein the double-stranded DNA synthesized in step (2) was inserted between two BsaI enzyme cleavage sites of the recombinant plasmid AtU6-26-sgRNA-SK, that is, obtaining the recombinant plasmid containing target fragment BRI1-T1, which was named recombinant plasmid AtU6-26-BRI1-T1-sgRNA.
(4) Double enzyme cleavage of the recombinant plasmid AtU6-26-sgRNA-SK was performed with restriction enzymes SpeI and NheI, and Fragment 7 of about 642 bp was recovered.
(5) Single enzyme cleavage of recombinant plasmid pYAO:Cas9 constructed in Example 1 was performed with restriction enzyme Spe I, and Vector Backbone 7 of about 14557 bp was recovered.
(6) Vector Backbone 7 was linked with Fragment 7, to obtain the recombinant plasmid pYAO: hspCas9-BRI1-sgRNA.

Via sequencing, the nucleotide sequence of the recombinant plasmid pYAO: hspCas9-BRI1-sgRNA is shown by SEQ ID NO: 21.

The recombinant plasmid pYAO: hspCas9-BRI1-sgRNA has one expression cassette II, the nucleotide sequence of which is like the double-stranded DNA molecule shown by Sites 8941-9575 (SEQ ID NO: 23) from 5′ terminal end in SEQ ID NO: 21, wherein Sites 8941-9388 (SEQ ID NO: 22) from 5′ terminal end in SEQ ID NO: 21 is AtU6-26 promoter, Sites 9390-9409 (SEQ ID NO: 24) is the nucleotide sequence of crRNA segment, Sites 9410-9485 (SEQ ID NO: 25) is the nucleotide sequence of tracrRNA segment, and Sites 9493-9575 (SEQ ID NO: 26) is the nucleotide sequence of 3′-UTR segment.

The pYAO promoter in the recombinant plasmid pYAO: hspCas9-BRI1-sgRNA was replaced with CaMV 35S promoter, to obtain the recombinant plasmid 35S: hspCas9-BRI1-sgRNA. The nucleotide sequence of CaMV 35S promoter is shown by (SEQ ID NO: 20).

### III). Transform and Preliminary Screening of Arabidopsis Thaliana

The recombinant plasmid (recombinant plasmid 35S:hSpCas9-BRI1-sgRNA or recombinant plasmid pYAO: hspCas9-BRI1-sgRNA) obtained in step II) was transformed into Agrobacterium tumefaciens GV3101 via electrotransformation (Gao Jianqiang, Liang Hua, Zhao Jun. Progress on the Floral-dip Method of Agrobacterium-mediated Plant Transformation, Chinese Agricultural Science Bulletin, 2010, 2 (16): 22-25), and the recombinant plasmid was then transformed into wild-type Arabidopsis thaliana by utilizing the method of Floral dip (reference: Zhang et al. Agrobacterium-mediated transformation of Arabidopsis thaliana using the floral dip method. Nat. Protoc. 2006.), so as to obtain the seeds of T₁ generation Arabidopsis thaliana.

The harvested seeds of T₁ generation Arabidopsis thaliana were screened in MS culture medium (containing 20 μg/L hygromycin and 150 μg/L carbenicillin), and 23 Arabidopsis thaliana plants of preliminary screening positive T₁ generation transfected with 35S:hSpCas9-BRI1-sgRNA and 21 Arabidopsis thaliana plants transfected with pYAO:hSpCas9-BRI1-sgRNA were obtained (non-positive transgenic Arabidopsis thaliana wilted and stopped growing, and substantially died after 15 days). 23 Arabidopsis thaliana plants of preliminary screening positive T₁ generation transfected with 35S:hSpCas9-BRI1-sgRNA were named 35S-1-T1, 35S-2-T1, 35S-3-T1, 35S-4-T1, 35S-5-T1, 35S-6-T1, 35S-7-T1, 35S-8-T1, 35S-9-T1, 35S-10-T1, 35S-11-T1, 35S-12-T1, 35S-13-T1, 35S-14-T1, 35S-15-T1, 35S-16-T1, 35S-17-T1, 35S-18-T1, 35S-19-T1, 35S-20-T1, 35S-21-T1, 35S-22-T1, and 35S-23-T1 in sequence, and 21 Arabidopsis thaliana plants of preliminary screening positive T₁ generation transfected with pYAO:hSpCas9-BRI1-sgRNA were named pYAO-1-T1, pYAO-2-T1, pYAO-3-T1, pYAO-4-T1, pYAO-5-T1, pYAO-6-T1, pYAO-7-T1, pYAO-8-T1, pYAO-9-T1, pYAO-10-T1, pYAO-11-T1, pYAO-12-T1, pYAO-13-T1, pYAO-14-T1, pYAO-15-T1, pYAO-16-T1, pYAO-17-T1, pYAO-18-T1, pYAO-19-T1, pYAO-20-T1, and pYAO-21-T1 in sequence.

Twenty-three (23) Arabidopsis thaliana plants of preliminary screening positive T₁ generation transfected with 35S:hSpCas9-BRI1-sgRNA and 21 Arabidopsis thaliana plants of preliminary screening positive T₁ generation transfected with pYAO:hSpCas9-BRI1-sgRNA were transferred into soil, and their phenotypes were observed.

The results show that, in 23 Arabidopsis thaliana plants transfected with 35S:hSpCas9-BRI1-sgRNA, only the phenotype of stunted plants occurred in 35S-5-T1, 35S-6-T1, 35S-8-T1, 35S-16-T1, and 35S-18-T1, and the phenotypes of the rest Arabidopsis thaliana plants have no significant difference from those of wild-type Arabidopsis thaliana. However, in 21 Arabidopsis thaliana plants transfected with pYAO:hSpCas9-BRI1-sgRNA, pYAO-5-T1, pYAO-7-T1, pYAO-11-T1, and pYAO-16-T1 only show as stunted plants, the phenotypes of pYAO-10-T1 and pYAO-12-T1 have no significant difference from those of wild-type Arabidopsis thaliana, the rest 15 plants show the similar phenotype as bri1 mutant, that is, stunted plant and contorted lamina.

IV). Analysis for the Editing Results of pYAO-Cas9/AtU6-26-sgRNA System to Endogenous Gene BRI1 of Arabidopsis thaliana Utilizing RFLP and PCR Products Sequencing
1. RFLP analysis for the editing results of endogenous gene BRI1 of Arabidopsis thaliana As the nucleotide sequence of target fragment BRI1-T1 contains a recognition sites of EcoR V, the editing results can be identified utilizing Restriction Fragment Length Polymorphism (RFLP). The PCR amplification products were obtained by the PCR amplification utilizing the genome DNAs extracted from the lamina of Arabidopsis thaliana plants of preliminary screening positive T₁ generation transfected with 35S:hSpCas9-BRI1-sgRNA and the lamina of Arabidopsis thaliana plants transfected with pYAO:hSpCas9-BRI1-sgRNA, respectively, as templates, and artificially synthesized BRI1-F: 5′-GATGGGATGAAGAAAGAGTG-3′(SEQ ID NO: 40) and BRI1-R: 5′-CTCATCTCTCTACCAACAAG-3′ (SEQ ID NO: 41) as primers. The recovered PCR amplification products were enzymatically cleaved with restriction enzyme EcoRV, and then were electrophoretically analyzed. As a control, the above experiments were performed using DNA of wild-type Arabidopsis thaliana as a template.

The results show that, in 23 35S:hSpCas9-BRI1-sgRNA transgenic Arabidopsis thaliana plants of T₁ generation, it was only detected that the editing of 35S-6-T1 with a phenotype of stunted plant occurred at selected target sites of BRI1 gene. However, in 21 pYAO:hSpCas9-BRI1-sgRNA transgenic plants of T1 generation, except that no editing results were detected in pYAO-10-T1 and pYAO-12-T1, editing occurred in all the rest 19 Arabidopsis thaliana plants at selected target sites of BRI1 gene.

2. Analysis for the Editing Results of Endogenous Gene BRI1 of Arabidopsis thaliana Utilizing PCR Products Sequencing

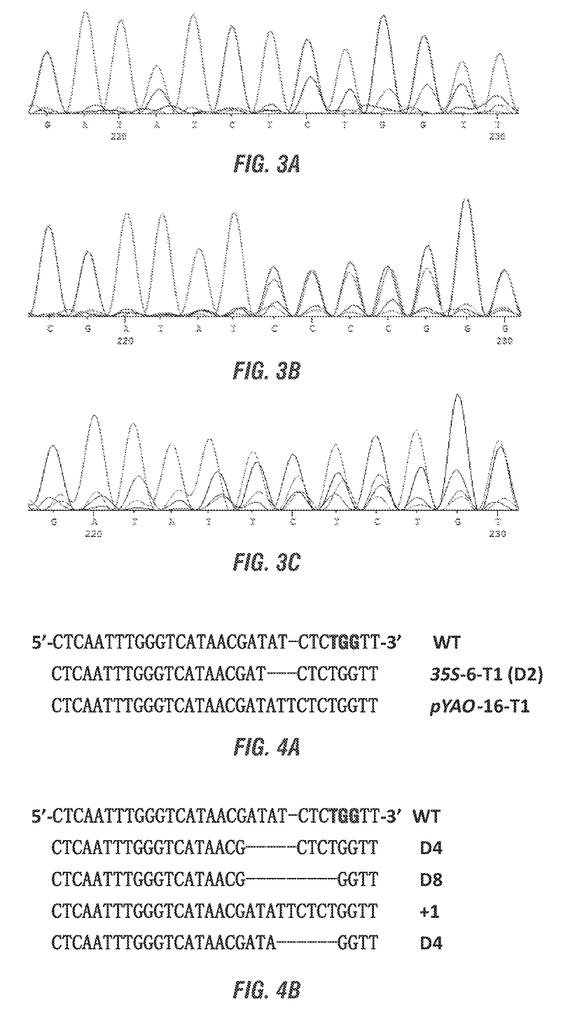

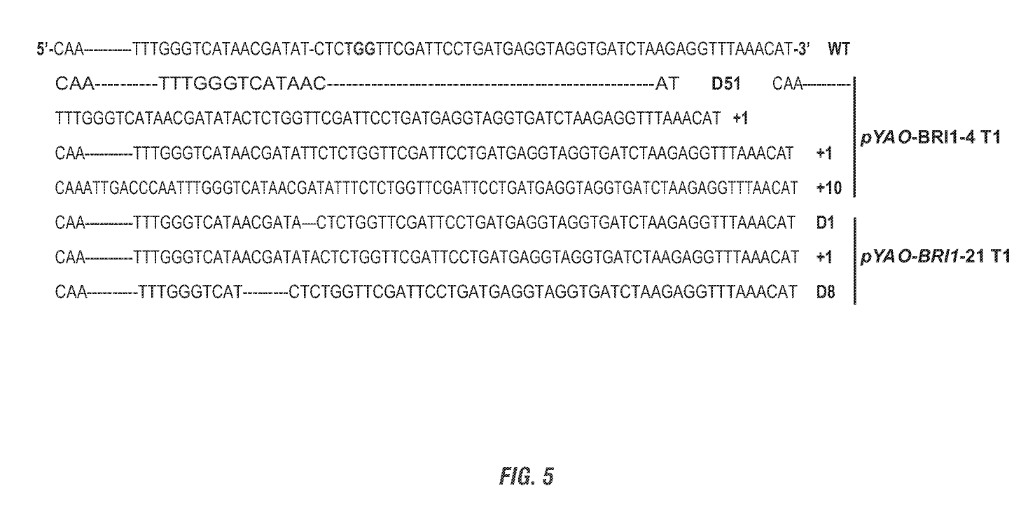

Sequencing analysis of the PCR products in step 1 was performed. The results show that (A in FIG. 3, B in FIG. 3 and A in FIG. 4), as for each of 35S-6-T1, pYAO-5-T1, pYAO-7-T1, pYAO-11-T1, and pYAO-16-T1, there were only two peaks at the selected target sites of BRI1 gene, and only one form of base insertion/deletion (indel) editing occurred.

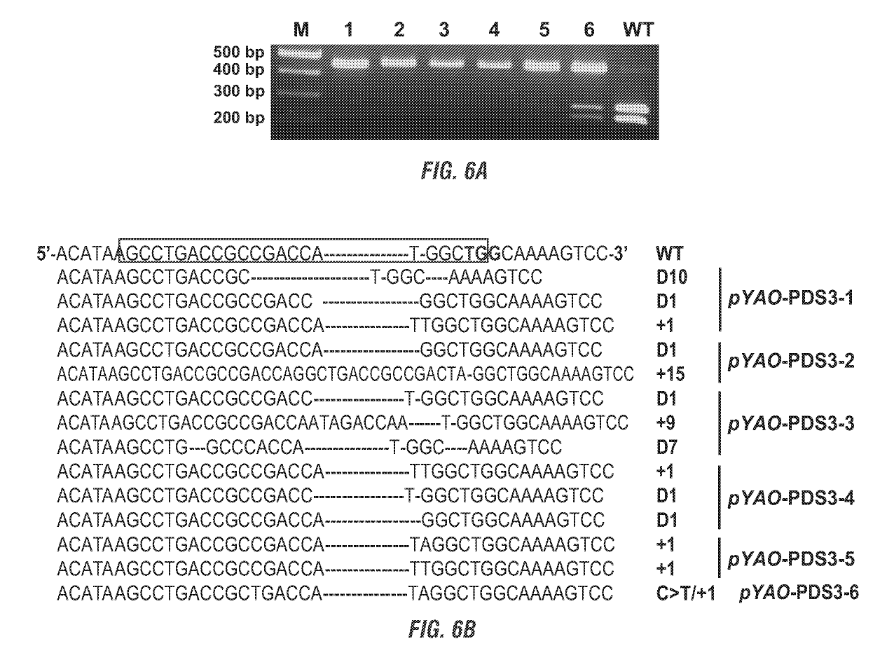

As for all 15 transgenic Arabidopsis thaliana plants with phenotypes of stunted plant and contorted lamina, there were multiple peaks at the selected target sites of BRI1 gene (C in FIG. 3), resulting in the editing forms at this target point can not be read. After the corresponding PCR products were recovered, and they were linked with pEASY-Blunt simple CloningVector (a product of Beijing TransGen Biotech Limited Corporation), and were sequenced. The sequencing results show that, as for 15 transgenic Arabidopsis thaliana plants with phenotypes of stunted plant and contorted lamina, there were multiple editing forms at the selected target sites of BRI1 gene (B in FIG. 4). Further, two pYAO:hSpCas9-BRI1-sgRNA T1 plant lines, which were similar to bri1 mutant, were analyzed by clone sequencing and multiple mutant alleles were detected in the BRI1 locus (FIG. 5).

Statistics of the site-directed editing efficiencies of 35S-Cas9/AtU6-26-sgRNA system and pYAO-Cas9/AtU6-26-sgRNA system for endogenous gene BRI1 of Arabidopsis thaliana were performed, statistics results are shown in Table 1, and the results show that, the editing efficiency of the Arabidopsis thaliana plants of T1 generation transfected with 35S:hSpCas9-BRI1-sgRNA is 4.3%, but the editing efficiency of the Arabidopsis thaliana plants of T1 generation transfected with pYAO:hSpCas9-BRI1-sgRNA is 90.5%. The results show that, editing efficiency of pYAO-Cas9/AtU6-26-sgRNA system for plant genomes is extremely significantly higher than that of 35S-Cas9/AtU6-26-sgRNA system.

TABLE 1
Statistics for the Editing Efficiencies of Site-directed
Editing Systems Initiated by Different Types of Promoters
for Endogenous Gene BRI1 of Arabidopsis thaliana

| | pYAO:hSpCas9- | 35S:hSpCas9- |
|---|---|---|
| | BRI1-sgRNA | BRI1-sgRNA |
| Positive transgenic sprouts of T1 | 21 | 23 |
| generation obtained by screening | | |
| Transgenic plants of T1 generation | 15 | 0 |
| shown as bri1 mutant phenotype | | |
| Transgenic plants of T1 generation | 19/21 (90.5%) | 1/23 (4.3%) |
| in which editing occurred at BRI1 | | |
| sites | | |

### Example 3 Analysis of Progeny Plants

Five plants showing the stunted phenotype with small seedling were segregated from T2 plants of a line of 35S:hSpCas9-BRI1-sgRNA edited at the BRI1 locus of progeny of T1 with several T2 lines having plants similar to the bri1 mutant phenotype at a low ratio (see Table 2 below).

TABLE 2
Segregation of T2 plants

| | | Phenotypic segregation of | | | | Phenotypic segregation of | |
|---|---|---|---|---|---|---|---|
| | | T2 plants | | | | T2 plants | |
| | | bril | Dwarf | | | bril | Dwarf |
| | T1 | phenotype/ | phenotype/ | | T1 | phenotype/ | phenotype/ |
| Line | phenotype | Total | Total | Line | phenotype | Total | Total |
| 35S-1 | Normal | 0/54 | 0/54 | pYAO-1 | Dwarf | 1/56 | 8/56 |
| 35S-3 | Normal | 0/50 | 0/50 | pYAO-2 | Dwarf | 0/21 | 1/21 |
| 35S-5 | Dwarf | 2/51 | 1/51 | pYAO-3 | Rosette | 42/49 | 3/49 |
| 35S-6 | Dwarf | 0/54 | 5/54 | pYAO-4 | Rosette | 31/49 | 3/49 |
| 35S-10 | Normal | 0/49 | 0/49 | pYAO-5 | Dwarf | 12/49 | 10/49 |
| 35S-12 | Normal | 0/46 | 2/46 | pYAO-7 | Dwarf | 43/56 | 0/56 |
| 35S-14 | Normal | 0/54 | 0/54 | pYAO-10 | Normal | 0/55 | 0/55 |
| 35S-16 | Dwarf | 3/55 | 7/55 | pYAO-12 | Normal | 0/56 | 0/56 |
| 35S-18 | Dwarf | 7/55 | 26/55 | pYAO-21 | Rosette | 18/46 | 22/46 |

T2 plants with the typical bri1 phenotype were obtained from the pYAO:hSpCas9-BRI1-sgRNA T1 plants. One T1 line had a mutant allele. In the T2 plants a few seedlings had a phenotype similar to the wild-type phenotype, however the T2 plants had a high segregation ratio of 76.3% or 43 out of 56 plants with the bri1 mutant phenotype. Seven plants had mutation at the BRI1 locus among 105 Cas9-free plants identified from the T2 progeny. The transmitting ratio is about 6.67%.
These results indicated that the genome editing by YAO promoter-based CRISPR/CAS9 system are successfully transmitted to the next generation.

### Example 4 Editing of PDS3 Gene

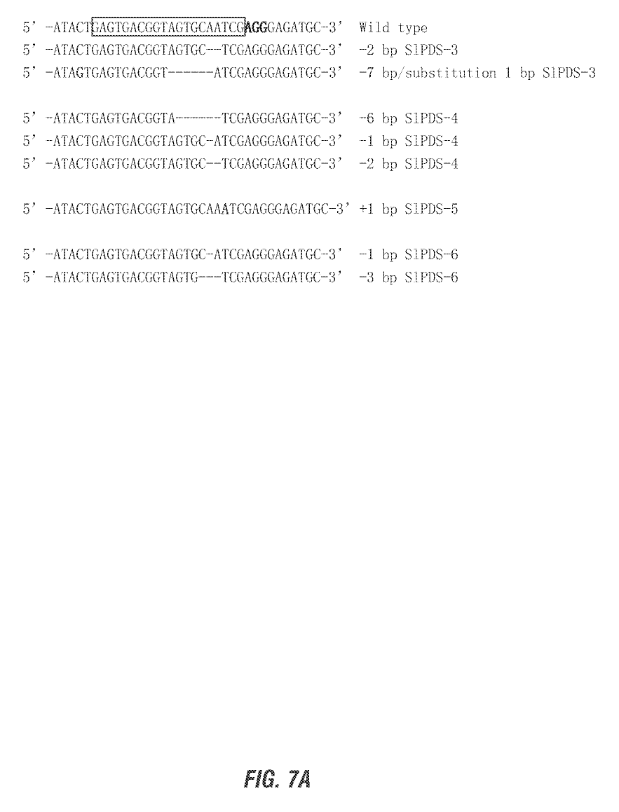

The PDS3 gene encodes a phytoene desaturase enzyme and catalyzes the desaturation of phytoene to zeta-carotene during carotenoid biosynthesis and the T-DNA insertion pds3 mutant exhibits albino and dwarf phenotypes (Qin et al., (2007) “Disruption of phytoene desaturase gene results in albino and dwarf phenotypes in Arabidopsis by impairing chlorophyll, carotenoid, and gibberellin biosynthesis” Cell Res. 17:471-482). pYAO:hSpCas9-PDS3-sgRNA was constructed and transformed into the wild-type Arabidopsis by floral dip method. Primer pairs P3 (5 ‘-TTACTGGTCAAGGCAAGACGATA-3 (SEQ ID NO: 42)’) and P4 (5′-AGTGAAAGCACATGCACGACA-3′ (SEQ ID NO: 43) were used for RFLP analysis. Twenty-three out of screened twenty-six transgenic T1 plants (88.5%) showed albino phenotypes at different degrees. RFLP analysis and DNA sequencing results suggested that the PDS3 locus was successfully edited (FIGS. 6A and 6B). The target sequence (SEQ ID NO: 44) is in the frame and the PAM sequence in bold.

### Example 5 Gene Editing of Tomato Genes

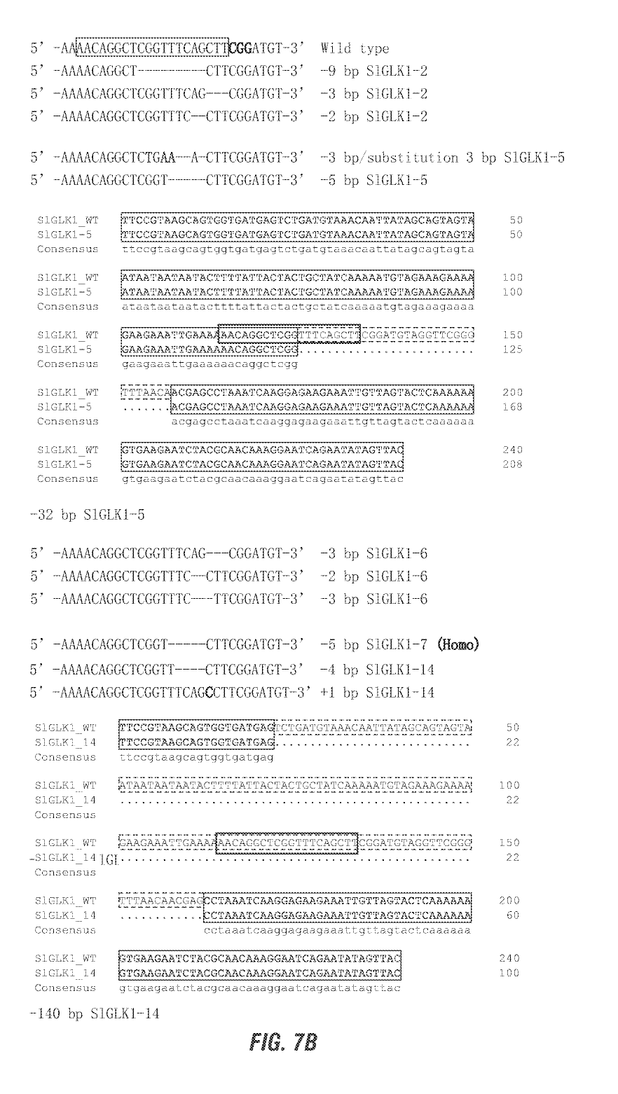

In order to measure the pYAO-driven CRISPR/Cas9 system would induce a high frequency of genome editing in crops, tomato genes SlPDS and SlGLK1 were selected to examine the efficiency of pYAO-driven CRISPR/Cas9 system in tomato. (See, for example, Nguyen et al. (2014) “Tomato GOLDEN2-LIKE transcription factors reveal molecular gradients that function during fruit development and ripening” Plant Cell 26(2):585-601. Eight T1 pYAO:Cas9-SlPDS3 transgenic plants were obtained. Only two of eight screened T1 pYAO:Cas9-SlPDS3 transgenic plants showed albino phenotypes. Statistical and DNA sequencing results suggested that the SlPDS3 locus of six T1 pYAO:Cas9-SlPDS3 transgenic plants was successfully edited and the ratios of T1 plants with the mutations was 75% (Table 3 and FIG. 7A).

TABLE 3
Statistical results of mutations in T1 pYAO:Cas9-SlPDS3 and
pYAO:Cas9-SlGLK1 transgenic plants of tomato.

| | NO. of T1 | NO. of T1 transgenic | The ratios of T1 |
|---|---|---|---|
| | transgenic | plants occurred | plants with the |
| | plants | mutation | mutations |
| pYAO: | 8 | 6 | 75% |
| Cas9- | | | |
| SlPDS3 | | | |
| pYAO: | 14 | 13 | 92.8% |
| Cas9- | | | |
| SlGLK1 | | | |

Meanwhile, fourteen T1 pYAO:Cas9-SlGLK1 transgenic plants were obtained and most of them exhibited the expected mosaic yellow leaves. Statistical results suggested that the SlGLK1 locus of thirteen T1 pYAO:Cas9-SlGLK1 transgenic plants was successfully edited and the ratios of T1 plants with the mutations was 92.8% (Table 3). As shown in FIG. 7B, the SlGLK1 locus of tomato genome occurred multi-forms editing, including knock outs of single nucleotide, multiple nucleotides, deletion large fragment, substitutions and insertions.

### Example 6 Editing of Maize Protoplasts

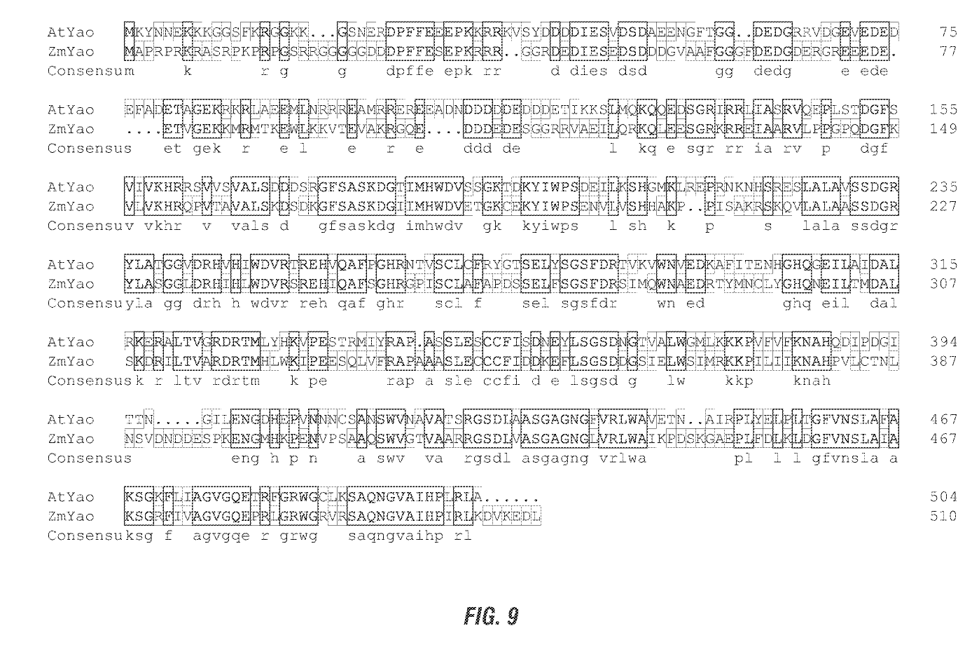

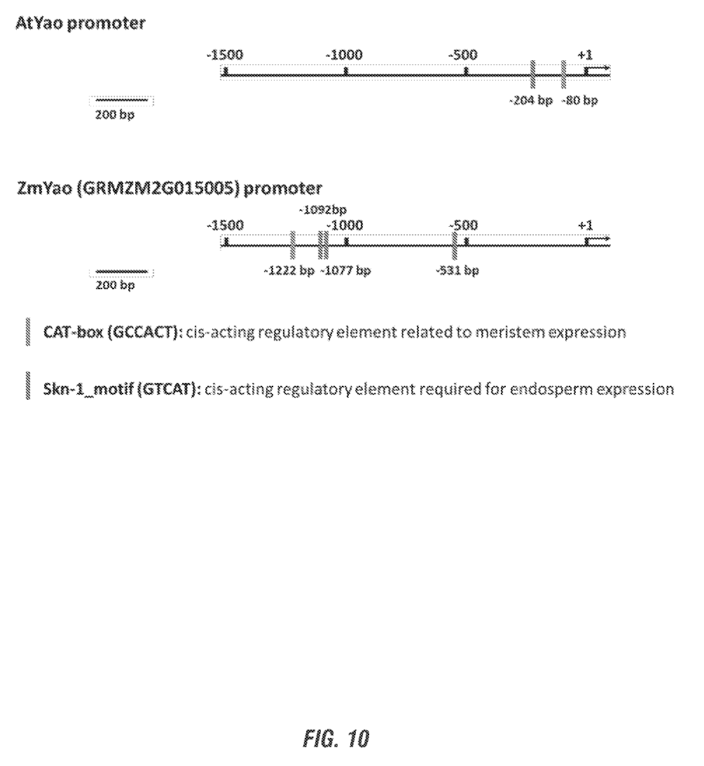

As YAO homologous genes exist in all eukaryotic organisms, the homolog of maize was found by a BLAST protocol and the promoter isolated to drive Cas9 expression as described above. The Arabidopsis (AtYao) homologous gene in Zea mays is predicted by Blastp. Its locus name is GRMZM2G015005 and the corresponding transcript name is GRMZM2G015005_T03. Here, this gene is named as ZmYao. The protein identity between AtYao and ZmYao is 51.82% (FIG. 9). In the original Yao paper (Li et al., 2013), the authors performed a pYAO::GUS-3U to monitor its expression pattern in plant tissues, and did not do any analysis about the promoter elements. Here, 982 bp fragment upstream from ATG start codon of AtYao (the same sequences as described in Li et al., 2013 and Yan et al., 2015 paper) was analyzed by PlantCARE software. Two interesting cis-acting regulatory elements were found: CAT-box and Skn-1 motif (FIG. 10). CAT-box (GCCACT) is related to meristem expression while Skn-1 motif (GTCAT) is required for endosperm expression. It is very likely that CAT-box and Skn-1 motif are associated with AtYao expression pattern. Meanwhile, similarity analysis was performed using 1, 500 bp fragment upstream from ATG start codon of ZmYao. As shown in FIG. 10, CAT-box and Skn-1 motif also existed in the ZmYao promoter (FIG. 10). This result indicated that the replacement of AtYao promoter by ZmYao promoter in the pYAO-driven CRISPR/Cas9 system is effective. Compared with pYAO-driven CRISPR/Cas9 system, the ZmYao promoter-driven CRISPR/Cas9 system was expected to have higher editing efficiency in monocot plants, such as rice and maize. Indeed the pYAO-driven CRISPR/Cas9 system showed edited result in maize protoplast. The ZmYAO promoter-driven CRISPR/Cas9 system was used to transform maize protoplasts. Using amplified PCR sequence as described above shows the locus of target genes were edited.

### Example 7 Editing of Rice Genome

OsPDS3 (LOC_Os03g08570) and OsSE5 (LOC_Os06g40080) were selected to confirm the genome editing efficiency of pYAO-driven CRISPR/Cas9 system in rice. Firstly, AtU6-26 promoter was replaced by OsU6a, which had been tested working well in rice by previously study (Ma et al., (2015) “A Robust CRISPR/Cas9 System for Convenient, High-Efficiency Multiplex Genome Editing in Monocot and Dicot Plants” Molecular Plant 8(8):1274-84). Then, pYAO:hSpCas9-OsPDS3-sgRNA and pYAO:hSpCas9-OsSE5-sgRNA were constructed and transformed into the callus of Nipponbare by Agrobacterium-mediated transformation. T1 transgenic plants were obtained and plants with mutant phenotype were identified and selected..

### Example 8 Use in TALENs and Zinc Finger Processes

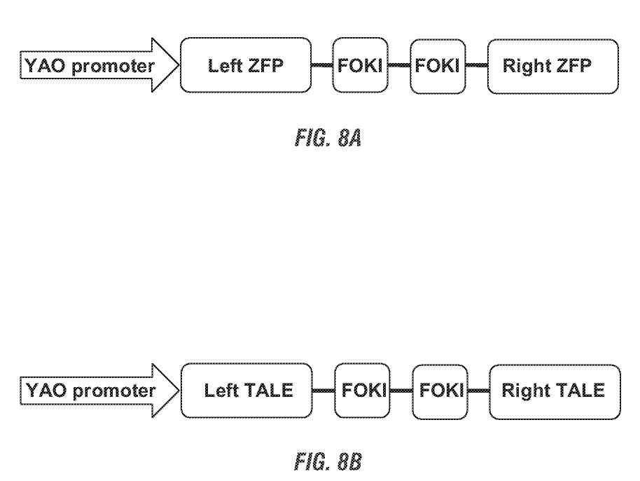

Only one promoter is needed for use in zinc finger nucleases (ZFNs) and in TALENs gene altering. A cassette is prepared for improving the gene editing efficiency of ZFNs and TALENs systems such as that shown in FIG. 8B and introduced into a plant cell using the methods described herein. For using in TALEN processes, the YAO promoter is operably linked to a first effector domain comprising TAL effector repeat sequences, a FokI endoculease, a second effector domain comprising TAL effector repeat sequence and a second FokI endonuclease. Similarity, the YAO promoter can also be in a zinc finger process and used to drive the Left ZFP-FOKI-FOKI-Right ZFP cassette expression as shown in FIG. 8B to increase the efficiency of regeneration.

### LIST OF SEQUENCES

SEQ ID 1 is expression cassette 1
SEQ ID NO: 2 is the YAO promoter, bases 1-1012 of SEQ ID NO: 1
SEQ ID NO: 3 is the Flag tag nucleotide sequences, bases 1019-1087 of SEQ ID NO: 1
SEQ ID NO: 4 is the nuclear localization signal I, bases 1088-1138 of SEQ ID NO: 1
SEQ ID NO: 5 is the Cas9 nuclease coding gene, bases 1139-5239 of SEQ ID NO: 1
SEQ ID NO: 6 is the nuclear localization signal II, bases 5240-5287 of SEQ ID NO: 1
SEQ ID NO: 7 is the NOS terminator, bases 5297-5580 of SEQ ID NO: 1
SEQ ID NO: 8 is the Cas9 nuclease
SEQ ID NO: 9 is the nucleotide sequence of sgRNA-F
SEQ ID NO: 10 is the nucleotide sequence of sgRNA-R
SEQ ID NO: 11 is the nucleotide sequence of 3′-UTR-F
SEQ ID NO: 12 is the nucleotide sequence of 3′-UTR-R
SEQ ID NO: 13 is the functional segment II of plasmid AtU6-26-sgRNA
SEQ ID NO: 14 is the AtU6-26 promoter, bases 1-448 of SEQ ID NO: 13
SEQ ID NO: 15 is the multiple cloning site segment, bases 449-471 of SEQ ID NO: 13
SEQ ID NO: 16 is and is a first enzyme cleavage site of BsaI, bases 451-456 of SEQ ID NO: 13
SEQ ID NO: 17 is and is a second cleavage site of BsaI, bases 465-470 of SEQ ID NO: 13
SEQ ID NO: 18 is and is the tracrRNA segment, bases 472-547 of SEQ ID NO: 13
SEQ ID NO: 19 is the 3′ UTR segment bases 555-637 of SEQ ID NO: 13
SEQ ID NO: 20 is the 35S promoter
SEQ ID NO: 21 is the plasmid pYAO: hspCas9-BRI1-sgRNA
SEQ ID NO: 22 is the AtU6-26 promoter, bases 8941-9388 of SEQ ID NO: 21
SEQ ID NO: 23 is the expression cassette II, bases 8941-9575 of SEQ ID NO: 21
SEQ ID NO: 24 is the crRNA segment bases, 9390-9409 of SEQ ID NO: 21
SEQ ID NO: 25 is the tracrRNA segment, bases, 9410-9485 of SEQ ID NO: 21
SEQ ID NO: 26 is the 3′-UTR segment, bases 9493-9575 of SEQ ID NO: 21
SEQ ID NO: 27 is the pYAO-F: primer
SEQ ID NO: 28 is the pYAO-R: primer
SEQ ID NO: 29 is the MCS-F primer
SEQ ID NO: 30 is the MCS-R primer
SEQ ID NO; 31 is the Amp^{r}BsaI-mutant-F primer
SEQ ID NO: 32 is the Amp^{r}BsaI-mutant-R primer
SEQ ID NO: 33 is the CS-F primer
SEQ ID NO: 34 is the CS-R primer
SEQ ID NO: 35 is the AtU6-26-F primer
SEQ ID NO: 36 is the AtU6-26-R primer
SEQ ID NO: 37 is the BRI1-T1 target fragment
SEQ ID NO: 38 is the BRI1-T1 F primer
SEQ ID NO: 39 is the BRI1-T1 R primer
SEQ ID NO: 40 is the BRI1-F primer
SEQ ID NO: 41 is the BRI1-R primer
SEQ ID NO: 42 is the P3 primer
SEQ ID NO: 43 is the P4 primer
SEQ ID NO: 44 is the target sequence of PDS3
SEQ ID NO: 45 is a region of the S1PDS wild type gene
SEQ ID NO: 46 is the modified region of −2 bp S1PDS-3 allele
SEQ ID NO: 47 is the modified region of the −7p 1 bp substation of S1PDS-3 allele
SEQ ID NO: 48 is the modified region of the −6 bp S1PDS-4 allele
SEQ ID NO: 49 is the modified region of the −1 bp S1PDS-4 allele
SEQ ID NO: 50 is the modified region of the −2 bp S1PDS-4 allele
SEQ ID NO: 51 is the modified region of +1 bp S1PDS-5 allele
SEQ ID NO: 52 is the modified region of the −1 bp S1PDS-6 allele
SEQ ID NO: 53 is the modified region of the −3 bp S1PDS-6 allele
SEQ ID NO: 54 is a region of the wild type SlGLK1-2 gene
SEQ ID NO: 55 is the modified region of—the 9 bp SlGLK1-2 allele
SEQ ID NO: 56 is the modified region of 3 bp SlGLK1-2 allele
SEQ ID NO: 57 is the modified region of −2 bp SlGLK1-2 allele
SEQ ID NO: 58 s the modified region of the −3 bp/substitution 3 bp SlGLK1-5 allele
SEQ ID NO: 59 is the modified region of the −5 bp SlGLK1-5 allele
SEQ ID NO: 60 is the aligned region of the S1GLK1 wild type sequence
SEQ ID NO: 61 is the aligned region of the SlGLK1-5 allele
SEQ ID NO: 62 is the consensus sequence of alignment of SlGLK1 wild type sequence and the −32 bp SlGLK1-5 allele
SEQ ID NO: 63 is the modified region of the −3 bp SlGLK1-6 allele
SEQ ID NO: 64 is the modified region of the −2 bp SlGLK1-6 allele
SEQ ID NO: 65 is another modified region of a −3 bp SlGLK1-6 allele
SEQ ID NO: 66 is the modified region of the −5 bp SlGLK1-7 (Homo)
SEQ ID NO: 67 is the modified region of the −4 bp SlGLK1-14 allele
SEQ ID NO: 68 is the modified region of the +1 bp SlGLK1-14 allele
SEQ ID NO: 69 is the aligned region of the S1LGK1 wild type gene aligned in FIG. 7
SEQ ID NO: 70 is the aligned region of the −140 bp SlGLK1-14 allele in FIG. 7
SEQ ID NO: 71 is the consensus sequence of the alignment of SEQ ID NO: 69 and 70
SEQ ID NO: 72 is a polypeptide encoded by an Arabidopsis YAO gene.
SEQ ID NO: 73 is a polypeptide encoded by a Zea mays YAO gene.
SEQ ID NO: 74 is the consensus sequence when aligning the Arabidopsis and Zea mays YAO polypeptide.

1. A method of altering a target nucleic acid molecule in a plant cell comprising, introducing into said cell a targeted nucleic acid molecule altering system comprising one or more expression cassettes comprising: a regulatory region of a YAO gene operably linked to at least one nucleotide sequence encoding a nuclease, whereby said target nucleic acid molecule in said cell is edited.

2. The method of claim 1 wherein said regulatory region of a YAO gene is selected from
(a) a regulatory region of a nucleotide sequence encoding a YAO polypeptide;
(b) a regulatory region comprising a homolog or ortholog of (a);
(c) a regulatory region of a nucleotide sequence encoding SEQ ID NO: 72 or SEQ ID NO: 73
(d) SEQ ID NO: 1;
(e) a regulatory region having at least 75% identity with SEQ ID NO: 1;
(f) a regulatory region hybridizing with the sequence of (c)-(e); or
(g) a functional fragment of (a)-(f).

3. The method of claim 1 wherein said homolog or ortholog comprises a CAT-box and Skn-1 motif.

4. The method of claim 1 wherein said regulatory region has at least 95% identity with SEQ ID NO: 1.

5. The method of claim 1 further comprising introducing said targeted nucleic acid molecule altering system into more than one plant cell, measuring the number of plant cells comprising said edited target nucleic acid molecule, wherein the number of plant cells comprising said edited target nucleic acid molecule is higher than the number of plant cells comprising said target edited nucleic acid molecule when said regulatory region is a 35S promoter.

6. The method of claim 1, further comprising introducing said nucleic acid molecule altering system into at least one plant cell, producing more than one plant, and measuring the number of plants comprising said edited target nucleic acid molecule, wherein at least 75% of said plants comprise said edited target nucleic acid molecule.

7. The method of claim 1, further comprising introducing said targeted nucleic acid molecule altering system into at least one plant cell, producing more than one plant, and measuring the number of plants comprising said edited target nucleic acid molecule, wherein at least 90% of said plants comprise said edited target nucleic acid molecule.

8. The method of claim 1, said system comprising a non naturally occurring Clustered Regularly Interspaced Short Palindormic Repeats (CRISPR) CRISPR associated (Cas) system comprising one or more expression cassettes comprising a) a first regulatory region operably linked to at least one nucleotide sequence encoding a CRISPR Cas system guide RNA that hybridizes with the target sequence, and b) a second regulatory region comprising said YAO regulatory region operably linked to a nucleotide sequence encoding a Cas9 nucleases wherein components (a) and (b) are located on the same or different vectors.

9. The method of claim 8, wherein a nucleic acid molecule is inserted at the locus of said target nucleic acid molecule.

10. The method of claim 8, further comprising introducing into said plant a second cassette comprising a single guide RNA (sgRNA) operably linked to a promoter.

11. The method of claim 10, wherein said promoter operable linked to said sgRNA comprises an AtU6-26 promoter.

12. The method of claim 8, the method further comprising introducing into said plant cell a cassette comprising a CRISPR RNA (crRNA) and a trans-encoded small RNA (tracrRNA) operably linked to a promoter and producing cleavage at said target nucleic acid molecule.

13. The method of claim 1, said system comprising a Transcription Activator-Like Effector Nucleases (TALEN) system, comprising one or more expression cassettes comprising said YAO regulatory region operably linked to at least one transcription activator-like (TAL) effector repeat sequences and a nuclease-encoding sequence, and producing a fusion protein, said fusion protein capable of binding said target nucleic acid molecule.

14. The method of claim 13, comprising said YAO regulatory region operably linked to a first TAL effector domain comprising TAL effector repeat sequences and a first nuclease-encoding sequence, a second TAL effector domain comprising TAL effector repeat sequences and a second-nuclease encoding sequence.

15. The method of claim 1, said system comprising a zinc finger nuclease system, comprising at least one expression cassette comprising said YAO promoter operably linked to at least one zinc finger protein binding said target nucleic acid molecule and a nuclease.

16. The method of claim 1, further comprising producing a plant comprising said edited target nucleic acid molecule, crossing said plant with a second plant and producing progeny comprising said edited target nucleic acid molecule.

17. The method of claim 16, further comprising producing more than one of said progeny, measuring the number of progeny comprising said edited target nucleic acid molecule, wherein at least at least 75% of said progeny segregate comprising said edited target nucleic acid molecule.

18.-20. (canceled)

21. An expression cassette comprising a regulatory region of a YAO gene operably linked to a nucleotide sequence encoding a Cas9 nuclease, said regulatory region selected from,
(a) a regulatory region of a nucleotide sequence encoding a YAO polypeptide;
(b) a regulatory region comprising a homolog or ortholog of (a);
(c) a regulatory region of a nucleotide sequence encoding SEQ ID NO: 72 or SEQ ID NO: 73
(d) SEQ ID NO: 1;
(e) a regulatory region having at least 75% identity with SEQ ID NO: 1;
(f) a regulatory region hybridizing with the sequence of (c)-(e); or
(g) a functional fragment of (a)-(f).

22. A vector comprising the expression cassette of claim 21.

23. A plant comprising an altered target nucleic acid molecule produced by the method of claim 1.

24.-25. (canceled)


SEQUENCE LISTING (114 sequences)

SEQ ID NO: 1 - DNA, 5580 bp, Artificial Sequence
  Description of Artificial Sequence Synthetic polynucleotide
gatgggaaattcattgaaaaccctaaacccaaatcaacagctgcaattcaaaaggggactaattgacaaacaaaaattgataacaaatagaggtagggggagagtttcgtacgcgacaatgagattgagctcttgaggacttgtgaagttgccaacgcacgagtgagtgacactggtcggtttgtgagccgtaacaacgtagttccatgagctcatcttcctcttctttgtctccagggaatttgagttcgactttctacgcgagggccctcgaggaagcttctagatttctgaatcgagctttcggaattttaacatagagaagttagagagagaatgaaaagccaaaggaggcgaaaatcgaacaaggaagaagaaagacaactttcgacaaagactggtcggtcggttttggtagacaattgaaattagatggatggtccggttcggtatactataagattaaaaacagttttaaattcagctaaaccgaactcatttgattttattaaaccggaatcatccgattcgagtttgtaaaaaataccgaaattgaaaacactaaacaaaaactgtattaaactgttactgaaataagagaatctcccaattcggtttacgtactactcttcagaaatcagaaccaaaaattcagaaatcggattgaaccaaacttaaattgacggtccggttagtcttcggctctacaaattaaaggcccaagtttctgctttaaaagaacgaaatagttaatgggctcaaaccatagaccaggtaagtcatgggcttggttagtccgggtcaacccggtagacccgattcctgaagaaaacctagtggaaggtttaaagttgtaaactttccgaccaaataaacaaaatcgttttccagcttcttccgtcgccactaaaccctgaggctaaacctagacgagtcaaagtgtaaaatcgttaaaccctaagagggagtgagagagagaagaatgaagtacaacaacgagaagaagaaaggagtcgagatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaaggatcctgattgatcgatagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgg

SEQ ID NO: 2 - DNA, 1012 bp, Artificial Sequence
  Description of Artificial Sequence Synthetic polynucleotide
gatgggaaattcattgaaaaccctaaacccaaatcaacagctgcaattcaaaaggggactaattgacaaacaaaaattgataacaaatagaggtagggggagagtttcgtacgcgacaatgagattgagctcttgaggacttgtgaagttgccaacgcacgagtgagtgacactggtcggtttgtgagccgtaacaacgtagttccatgagctcatcttcctcttctttgtctccagggaatttgagttcgactttctacgcgagggccctcgaggaagcttctagatttctgaatcgagctttcggaattttaacatagagaagttagagagagaatgaaaagccaaaggaggcgaaaatcgaacaaggaagaagaaagacaactttcgacaaagactggtcggtcggttttggtagacaattgaaattagatggatggtccggttcggtatactataagattaaaaacagttttaaattcagctaaaccgaactcatttgattttattaaaccggaatcatccgattcgagtttgtaaaaaataccgaaattgaaaacactaaacaaaaactgtattaaactgttactgaaataagagaatctcccaattcggtttacgtactactcttcagaaatcagaaccaaaaattcagaaatcggattgaaccaaacttaaattgacggtccggttagtcttcggctctacaaattaaaggcccaagtttctgctttaaaagaacgaaatagttaatgggctcaaaccatagaccaggtaagtcatgggcttggttagtccgggtcaacccggtagacccgattcctgaagaaaacctagtggaaggtttaaagttgtaaactttccgaccaaataaacaaaatcgttttccagcttcttccgtcgccactaaaccctgaggctaaacctagacgagtcaaagtgtaaaatcgttaaaccctaagagggagtgagagagagaagaatgaagtacaacaacgagaagaagaaagga

SEQ ID NO: 3 - DNA, 69 bp, Artificial Sequence
  Description of Artificial Sequence Synthetic oligonucleotide
atggactataaggaccacgacggagactacaaggatcatgatattgattacaaagacgatgacgataag

SEQ ID NO: 4 - DNA, 51 bp, Artificial Sequence
  Description of Artificial Sequence Synthetic oligonucleotide
atggccccaaagaagaagcggaaggtcggtatccacggagtcccagcagcc

SEQ ID NO: 5 - DNA, 4101 bp, Artificial Sequence
  Description of Artificial Sequence Synthetic polynucleotide
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 6 - DNA, 48 bp, Artificial Sequence
  Description of Artificial Sequence Synthetic oligonucleotide
aaaaggccggcggccacgaaaaaggccggccaggcaaaaaagaaaaag

SEQ ID NO: 7 - DNA, 284 bp, Artificial Sequence
  Description of Artificial Sequence Synthetic polynucleotide
tgattgatcgatagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgg

SEQ ID NO: 8 - protein, 1367 aa, Artificial Sequence
  Description of Artificial Sequence Synthetic polypeptide
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 9 - DNA, 91 bp, Artificial Sequence
  Description of Artificial Sequence Synthetic primer
aattcaggtctcagttttagagctagaaatagcaagttaaaataaggctagtccgttatcaacttgaaaaagtggcaccgagtcggtgctt

SEQ ID NO: 10 - DNA, 95 bp, Artificial Sequence
  Description of Artificial Sequence Synthetic primer
ggcaaaaaaagcaccgactcggtgccactttttcaagttgataacggactagccttattttaacttgctatttctagctctaaaactgagacctg

SEQ ID NO: 11 - DNA, 88 bp, Artificial Sequence
  Description of Artificial Sequence Synthetic oligonucleotide
tttttgccattcttttcaagctccattgtcaaattttcggggggttttgaagtcgcctatctgaggttagtctctctgcatctgatca

SEQ ID NO: 12 - DNA, 84 bp, Artificial Sequence
  Description of Artificial Sequence Synthetic oligonucleotide
ctagtgatcagatgcagagagactaacctcagataggcgacttcaaaaccccccgaaaatttgacaatggagcttgaaaagaat

SEQ ID NO: 13 - DNA, 637 bp, Artificial Sequence
  Description of Artificial Sequence Synthetic polynucleotide
aagcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtgagacctgaattcaggtctcagttttagagctagaaatagcaagttaaaataaggctagtccgttatcaacttgaaaaagtggcaccgagtcggtgctttttttgccattcttttcaagctccattgtcaaattttcggggggttttgaagtcgcctatctgaggttagtctctctgcatctgatca

SEQ ID NO: 14 - DNA, 448 bp, Artificial Sequence
  Description of Artificial Sequence Synthetic polynucleotide
aagcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgatt

SEQ ID NO: 15 - DNA, 23 bp, Artificial Sequence
  Description of Artificial Sequence Synthetic oligonucleotide
gtgagacctgaattcaggtctca

SEQ ID NO: 16 - DNA, 6 bp, Artificial Sequence
  Description of Artificial Sequence Synthetic oligonucleotide
gagacc

SEQ ID NO: 17 - DNA, 6 bp, Artificial Sequence
  Description of Artificial Sequence Synthetic oligonucleotide
ggtctc

SEQ ID NO: 18 - DNA, 76 bp, Artificial Sequence
  Description of Artificial Sequence Synthetic oligonucleotide
gttttagagctagaaatagcaagttaaaataaggctagtccgttatcaacttgaaaaagtggcaccgagtcggtgc

SEQ ID NO: 19 - DNA, 83 bp, Artificial Sequence
  Description of Artificial Sequence Synthetic oligonucleotide
gccattcttttcaagctccattgtcaaattttcggggggttttgaagtcgcctatctgaggttagtctctctgcatctgatca

SEQ ID NO: 20 - DNA, 902 bp, Artificial Sequence
  Description of Artificial Sequence Synthetic polynucleotide
gagctcatagcaagcatttaccacttttaaaagtcttttaacgaaaacgaaattttctttactaaatttaacgacgttatcttcatcttacgaactaacgaactctaagcaaacaaaacatatacaacacaactccagctccaggagaggtttactttacttgaaggaatatatctccttcccagaacgcttcctatcaccctaacacgcagtagggaatgcagtcacctctatagtgtagttaggtgaacgaaacttctgcaccaaccttgcagaagaaaaaggtgctacgaggagcacccacccccaggtagaaaccctggtgacagccgtctccgtagaagttgctaccggaaaggaaatagcgttactaccgtaaacatcctcggtggaaggaaaaggtgatagaagtgttatttcactgtctatcgacccgttaccttaggctcctccaaaggcctatagtgggaaacaacttttcagagttaacgggaaaccagaagactctgacatagaaactataaaaacctcatctgttcacacagcacgaggtggtacaatagtgtagttaggtgaacgaaacttctgcaccaaccttgcagaagaaaaaggtgctacgaggagcacccacccccaggtagaaaccctggtgacagccgtctccgtagaagttgctaccggaaaggaaatagcgttactaccgtaaacatcctcggtggaaggaaaaggtgatagaagtgttatttcactgtctatcgacccgttaccttaggctcctccaaaggcctataatgggaaacaacttttcagagttaacgggaaaccagaagactctgacatagaaactataaaaacctcatctgttcacacagcacgaggtggtacaactggacgtccgtacg

SEQ ID NO: 21 - DNA, 15197 bp, Artificial Sequence
  Description of Artificial Sequence Synthetic polynucleotide
gtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcgactctagatcactagcaagcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgttgggtcataacgatatctcgttttagagctagaaatagcaagttaaaataaggctagtccgttatcaacttgaaaaagtggcaccgagtcggtgctttttttgccattcttttcaagctccattgtcaaattttcggggggttttgaagtcgcctatctgaggttagtctctctgcatctgatcactagtatcctaggaaggtaccgggccccccctcgacgatgggaaattcattgaaaaccctaaacccaaatcaacagctgcaattcaaaaggggactaattgacaaacaaaaattgataacaaatagaggtagggggagagtttcgtacgcgacaatgagattgagctcttgaggacttgtgaagttgccaacgcacgagtgagtgacactggtcggtttgtgagccgtaacaacgtagttccatgagctcatcttcctcttctttgtctccagggaatttgagttcgactttctacgcgagggccctcgaggaagcttctagatttctgaatcgagctttcggaattttaacatagagaagttagagagagaatgaaaagccaaaggaggcgaaaatcgaacaaggaagaagaaagacaactttcgacaaagactggtcggtcggttttggtagacaattgaaattagatggatggtccggttcggtatactataagattaaaaacagttttaaattcagctaaaccgaactcatttgattttattaaaccggaatcatccgattcgagtttgtaaaaaataccgaaattgaaaacactaaacaaaaactgtattaaactgttactgaaataagagaatctcccaattcggtttacgtactactcttcagaaatcagaaccaaaaattcagaaatcggattgaaccaaacttaaattgacggtccggttagtcttcggctctacaaattaaaggcccaagtttctgctttaaaagaacgaaatagttaatgggctcaaaccatagaccaggtaagtcatgggcttggttagtccgggtcaacccggtagacccgattcctgaagaaaacctagtggaaggtttaaagttgtaaactttccgaccaaataaacaaaatcgttttccagcttcttccgtcgccactaaaccctgaggctaaacctagacgagtcaaagtgtaaaatcgttaaaccctaagagggagtgagagagagaagaatgaagtacaacaacgagaagaagaaaggagtcgagatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaaggatcctgattgatcgatagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattc

SEQ ID NO: 22 - DNA, 448 bp, Artificial Sequence; identical to SEQ ID NO: 14
  Description of Artificial Sequence Synthetic polynucleotide

SEQ ID NO: 23 - DNA, 635 bp, Artificial Sequence
  Description of Artificial Sequence Synthetic polynucleotide
aagcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgttgggtcataacgatatctcgttttagagctagaaatagcaagttaaaataaggctagtccgttatcaacttgaaaaagtggcaccgagtcggtgctttttttgccattcttttcaagctccattgtcaaattttcggggggttttgaagtcgcctatctgaggttagtctctctgcatctgatca

SEQ ID NO: 24 - DNA, 20 bp, Artificial Sequence
  Description of Artificial Sequence Synthetic oligonucleotide
ttgggtcataacgatatctc

SEQ ID NO: 25 - DNA, 76 bp, Artificial Sequence; identical to SEQ ID NO: 18
  Description of Artificial Sequence Synthetic oligonucleotide

SEQ ID NO: 26 - DNA, 83 bp, Artificial Sequence; identical to SEQ ID NO: 19
  Description of Artificial Sequence Synthetic oligonucleotide

SEQ ID NO: 27 - DNA, 32 bp, Artificial Sequence
  Description of Artificial Sequence Synthetic primer
aagtcgacgatgggaaattcattgaaaaccct

SEQ ID NO: 28 - DNA, 31 bp, Artificial Sequence
  Description of Artificial Sequence Synthetic primer
aagtcgactcctttcttcttctcgttgttgt

SEQ ID NO: 29 - DNA, 28 bp, Artificial Sequence
  Description of Artificial Sequence Synthetic primer
ctagatcactagtatcctaggaaggtac

SEQ ID NO: 30 - DNA, 20 bp, Artificial Sequence
  Description of Artificial Sequence Synthetic primer
cttcctaggatactagtgat

SEQ ID NO: 31 - DNA, 40 bp, Artificial Sequence
  Description of Artificial Sequence Synthetic primer
ggccccagtgctgcaatgataccgcgcgacccacgctcac

SEQ ID NO: 32 - DNA, 40 bp, Artificial Sequence
  Description of Artificial Sequence Synthetic primer
gtgagcgtgggtcgcgcggtatcattgcagcactggggcc

SEQ ID NO: 33 - DNA, 44 bp, Artificial Sequence
  Description of Artificial Sequence Synthetic primer
cactatagggcgaattgggtgctagcccccccctcgaggtcgac

SEQ ID NO: 34 - DNA, 44 bp, Artificial Sequence
  Description of Artificial Sequence Synthetic primer
gtcgacctcgaggggggggctagcacccaattcgccctatagtg

SEQ ID NO: 35 - DNA, 33 bp, Artificial Sequence
  Description of Artificial Sequence Synthetic primer
aagctagcaagcttcgttgaacaacggaaactc

SEQ ID NO: 36 - DNA, 41 bp, Artificial Sequence
  Description of Artificial Sequence Synthetic primer
aagaattcaggtctcacaatcactacttcgactctagctgt

SEQ ID NO: 37 - DNA, 20 bp, Artificial Sequence
  Description of Artificial Sequence Synthetic primer
ttgggtcataacgatatctc

SEQ ID NO: 38 - DNA, 24 bp, Artificial Sequence
  Description of Artificial Sequence Synthetic primer
attgttgggtcataacgatatctc

SEQ ID NO: 39 - DNA, 24 bp, Artificial Sequence
  Description of Artificial Sequence Synthetic primer
aaacgagatatcgttatgacccaa

SEQ ID NO: 40 - DNA, 20 bp, Artificial Sequence
  Description of Artificial Sequence Synthetic primer
gatgggatgaagaaagagtg

SEQ ID NO: 41 - DNA, 20 bp, Artificial Sequence
  Description of Artificial Sequence Synthetic primer
ctcatctctctaccaacaag

SEQ ID NO: 42 - DNA, 23 bp, Artificial Sequence
  Description of Artificial Sequence Synthetic primer
ttactggtcaaggcaagacgata

SEQ ID NO: 43 - DNA, 21 bp, Artificial Sequence
  Description of Artificial Sequence Synthetic primer
agtgaaagcacatgcacgaca

SEQ ID NO: 44 - DNA, 22 bp, Artificial Sequence
  Description of Artificial Sequence Synthetic oligonucleotide
gcctgaccgccgaccatggctg

SEQ ID NO: 45 - DNA, 35 bp, Solanum lycopersicum
atactgagtgacggtagtgcaatcgagggagatgc

SEQ ID NO: 46 - DNA, 33 bp, Artificial Sequence
  Description of Artificial Sequence Synthetic oligonucleotide
atactgagtgacggtagtgctcgagggagatgc

SEQ ID NO: 47 - DNA, 29 bp, Artificial Sequence
  Description of Artificial Sequence Synthetic oligonucleotide
atagtgagtgacggtatcgagggagatgc

SEQ ID NO: 48 - DNA, 29 bp, Artificial Sequence
  Description of Artificial Sequence Synthetic oligonucleotide
atactgagtgacggtatcgagggagatgc

SEQ ID NO: 49 - DNA, 34 bp, Artificial Sequence
  Description of Artificial Sequence Synthetic oligonucleotide
atactgagtgacggtagtgcatcgagggagatgc

SEQ ID NO: 50 - DNA, 33 bp, Artificial Sequence; identical to SEQ ID NO: 46
  Description of Artificial Sequence Synthetic oligonucleotide

SEQ ID NO: 51 - DNA, 36 bp, Artificial Sequence
  Description of Artificial Sequence Synthetic oligonucleotide
atactgagtgacggtagtgcaaatcgagggagatgc

SEQ ID NO: 52 - DNA, 34 bp, Artificial Sequence; identical to SEQ ID NO: 49
  Description of Artificial Sequence Synthetic oligonucleotide

SEQ ID NO: 53 - DNA, 32 bp, Artificial Sequence
  Description of Artificial Sequence Synthetic oligonucleotide
atactgagtgacggtagtgtcgagggagatgc

SEQ ID NO: 54 - DNA, 29 bp, Artificial Sequence
  Description of Artificial Sequence Synthetic oligonucleotide
aaaacaggctcggtttcagcttcggatgt

SEQ ID NO: 55 - DNA, 20 bp, Artificial Sequence
  Description of Artificial Sequence Synthetic oligonucleotide
aaaacaggctcttcggatgt

SEQ ID NO: 56 - DNA, 26 bp, Artificial Sequence
  Description of Artificial Sequence Synthetic oligonucleotide
aaaacaggctcggtttcagcggatgt

SEQ ID NO: 57 - DNA, 27 bp, Artificial Sequence
  Description of Artificial Sequence Synthetic oligonucleotide
aaaacaggctcggtttccttcggatgt

SEQ ID NO: 58 - DNA, 26 bp, Artificial Sequence
  Description of Artificial Sequence Synthetic oligonucleotide
aaaacaggctctgaaacttcggatgt

SEQ ID NO: 59 - DNA, 24 bp, Artificial Sequence
  Description of Artificial Sequence Synthetic oligonucleotide
aaaacaggctcggtcttcggatgt

SEQ ID NO: 60 - DNA, 240 bp, Solanum lycopersicum
ttccgtaagcagtggtgatgagtctgatgtaaacaattatagcagtagtaataataataatacttttattactactgctatcaaaaatgtagaaagaaaagaagaaattgaaaaaacaggctcggtttcagcttcggatgtaggttcgggtttaacaacgagcctaaatcaaggagaagaaattgttagtactcaaaaaagtgaagaatctacgcaacaaaggaatcagaatatagttac

SEQ ID NO: 61 - DNA, 208 bp, Artificial Sequence
  Description of Artificial Sequence Synthetic polynucleotide
ttccgtaagcagtggtgatgagtctgatgtaaacaattatagcagtagtaataataataatacttttattactactgctatcaaaaatgtagaaagaaaagaagaaattgaaaaaacaggctcggacgagcctaaatcaaggagaagaaattgttagtactcaaaaaagtgaagaatctacgcaacaaaggaatcagaatatagttac

SEQ ID NO: 62 - DNA, 240 bp, Artificial Sequence; identical to SEQ ID NO: 60
  Description of Artificial Sequence Synthetic consensus sequence
  misc_feature (126)..(157): May or may not be present

SEQ ID NO: 63 - DNA, 26 bp, Artificial Sequence
  Description of Artificial Sequence Synthetic oligonucleotide
aaaacaggctcggtttcagcggatgt

SEQ ID NO: 64 - DNA, 27 bp, Artificial Sequence
  Description of Artificial Sequence Synthetic oligonucleotide
aaaacaggctcggtttccttcggatgt

SEQ ID NO: 65 - DNA, 26 bp, Artificial Sequence
  Description of Artificial Sequence Synthetic oligonucleotide
aaaacaggctcggtttcttcggatgt

SEQ ID NO: 66 - DNA, 24 bp, Artificial Sequence
  Description of Artificial Sequence Synthetic oligonucleotide
aaaacaggctcggtcttcggatgt

SEQ ID NO: 67 - DNA, 25 bp, Artificial Sequence
  Description of Artificial Sequence Synthetic oligonucleotide
aaaacaggctcggttcttcggatgt

SEQ ID NO: 68 - DNA, 30 bp, Artificial Sequence
  Description of Artificial Sequence Synthetic oligonucleotide
aaaacaggctcggtttcagccttcggatgt

SEQ ID NO: 69 - DNA, 240 bp, Solanum lycopersicum; identical to SEQ ID NO: 60

SEQ ID NO: 70 - DNA, 100 bp, Artificial Sequence
  Description of Artificial Sequence Synthetic polynucleotide
ttccgtaagcagtggtgatgagcctaaatcaaggagaagaaattgttagtactcaaaaaagtgaagaatctacgcaacaaaggaatcagaatatagttac

SEQ ID NO: 71 - DNA, 240 bp, Artificial Sequence; identical to SEQ ID NO: 60
  Description of Artificial Sequence Synthetic consensus sequence
  misc_feature (23)..(162): May or may not be present

SEQ ID NO: 72 - protein, 504 aa, Arabidopsis thaliana
MKYNNEKKKGGSFKRGGKKGSNERDPFFEEEPKKRRKVSYDDDDIESVDSDAEENGFTGGDEDGRRVDGEVEDEDEFADETAGEKRKRLAEEMLNRRREAMRREREEADNDDDDDEDDDETIKKSLMQKQQEDSGRIRRLIASRVQEPLSTDGFSVIVKHRRSVVSVALSDDDSRGFSASKDGTIMHWDVSSGKTDKYIWPSDEILKSHGMKLREPRNKNHSRESLALAVSSDGRYLATGGVDRHVHIWDVRTREHVQAFPGHRNTVSCLCFRYGTSELYSGSFDRTVKVWNVEDKAFITENHGHQGEILAIDALRKERALTVGRDRTMLYHKVPESTRMIYRAPASSLESCCFISDNEYLSGSDNGTVALWGMLKKKPVFVFKNAHQDIPDGITTNGILENGDHEPVNNNCSANSWVNAVATSRGSDLAASGAGNGFVRLWAVETNAIRPLYELPLTGFVNSLAFAKSGKFLIAGVGQETRFGRWGCLKSAQNGVAIHPLRLA

SEQ ID NO: 73 - protein, 510 aa, Zea mays
MAPRPRKRASRPKPRPGSRRGGGGGDDDPFFESEPKRRRGGRDEDIESEDSDDDGVAAFGGGFDEDGDERGREEEDEETVGEKKMRMTKEWLKKVTEVAKRGQEDDDEDESGGRRVAEILQRKQLEESGRKRREIAARVLPPGPQDGFKVLVKHRQPVTAVALSKDSDKGFSASKDGIIMHWDVETGKCEKYIWPSENVLVSHHAKPPISAKRSKQVLALAASSDGRYLASGGLDRHIHLWDVRSREHIQAFSGHRGPISCLAFAPDSSELFSGSFDRSIMQWNAEDRTYMNCLYGHQNEILTMDALSKDRILTVARDRTMHLWKIPEESQLVFRAPAAASLECCCFIDDKEFLSGSDDGSIELWSIMRKKPILIIKNAHPVLCTNLNSVDNDDESPKENGMHKPENVPSAAQSWVGTVAARRGSDLVASGAGNGLVRLWAIKPDSKGAEPLFDLKLDGFVNSLAIAKSGRFIVAGVGQEPRLGRWGRVRSAQNGVAIHPIRLKDVKEDL

SEQ ID NO: 74 - protein, 523 aa, Artificial Sequence
  Description of Artificial Sequence Synthetic consensus sequence
  MOD_RES (2)..(2): Lys or Ala
  MOD_RES (3)..(3): Tyr or Pro
  MOD_RES (4)..(4): Asn or Arg
  MOD_RES (5)..(5): Asn or Pro
  MOD_RES (6)..(6): Glu or Arg
  MOD_RES (8)..(8): Lys or Arg
  MOD_RES (9)..(9): Lys or Ala
  MOD_RES (10)..(10): Gly or Ser
  MOD_RES (11)..(11): Gly or Arg
  MOD_RES (12)..(12): Ser or Pro
  MOD_RES (13)..(13): Phe or Lys
  MOD_RES (14)..(14): Lys or Pro
  MOD_RES (16)..(16): Gly or Pro
  MOD_RES (18)..(18): Lys or Ser
  MOD_RES (19)..(19): Lys or Arg
  MOD_RES (20)..(22): May or may not be present
  MOD_RES (24)..(24): Ser or Gly
  MOD_RES (25)..(25): Asn or Gly
  MOD_RES (26)..(26): Glu or Asp
  MOD_RES (27)..(27): Arg or Asp
  MOD_RES (33)..(33): Glu or Ser
  MOD_RES (37)..(37): Lys or Arg
  MOD_RES (40)..(41): May or may not be present
  MOD_RES (42)..(42): Ser or Gly
  MOD_RES (43)..(43): Tyr or Gly
  MOD_RES (44)..(44): Asp or Arg
  MOD_RES (46)..(46): Asp or Glu
  MOD_RES (51)..(51): Val or Glu
  MOD_RES (55)..(55): Ala or Asp
  MOD_RES (56)..(56): Glu or Asp
  MOD_RES (57)..(57): Glu or Gly
  MOD_RES (58)..(58): Asn or Val
  MOD_RES (59)..(59): Gly or Ala
  MOD_RES (60)..(60): Phe or Ala
  MOD_RES (61)..(61): Thr or Phe
  MOD_RES (64)..(65): May or may not be present
  MOD_RES (70)..(70): Arg or Asp
  MOD_RES (71)..(71): Arg or Glu
  MOD_RES (72)..(72): Val or Arg
  MOD_RES (73)..(73): Asp or Gly
  MOD_RES (74)..(74): Gly or Arg
  MOD_RES (76)..(76): Val or Glu
  MOD_RES (80)..(84): May or may not be present
  MOD_RES (87)..(87): Ala or Val
  MOD_RES (91)..(91): Arg or Lys
  MOD_RES (92)..(92): Lys or Met
  MOD_RES (94)..(94): Leu or Met
  MOD_RES (95)..(95): Ala or Thr
  MOD_RES (96)..(96): Glu or Lys
  MOD_RES (98)..(98): Met or Trp
  MOD_RES (100)..(100): Asn or Lys
  MOD_RES (101)..(101): Arg or Lys
  MOD_RES (102)..(102): Arg or Val
  MOD_RES (103)..(103): Arg or Thr
  MOD_RES (105)..(105): Ala or Val
  MOD_RES (106)..(106): Met or Ala
  MOD_RES (107)..(107): Arg or Lys
  MOD_RES (109)..(109): Glu or Gly
  MOD_RES (110)..(110): Arg or Gln
  MOD_RES (112)..(115): May or may not be present
  MOD_RES (119)..(119): Asp or Glu
  MOD_RES (122)..(122): Asp or Ser
  MOD_RES (123)..(124): Asp or Gly
  MOD_RES (125)..(125): Glu or Arg
  MOD_RES (126)..(126): Thr or Arg
  MOD_RES (127)..(127): Ile or Val
  MOD_RES (128)..(128): Lys or Ala
  MOD_RES (129)..(129): Lys or Glu
  MOD_RES (130)..(130): Ser or Ile
  MOD_RES (132)..(132): Met or Gln
  MOD_RES (133)..(133): Gln or Arg
  MOD_RES (136)..(136): Gln or Leu
  MOD_RES (138)..(138): Asp or Glu
  MOD_RES (142)..(142): Ile or Lys
  MOD_RES (145)..(145): Leu or Glu
  MOD_RES (148)..(148): Ser or Ala
  MOD_RES (151)..(151): Gln or Leu
  MOD_RES (152)..(152): Glu or Pro
  MOD_RES (154)..(154): Leu or Gly
  MOD_RES (155)..(155): Ser or Pro
  MOD_RES (156)..(156): Thr or Gln
  MOD_RES (160)..(160): Ser or Lys
  MOD_RES (162)..(162): Ile or Leu
  MOD_RES (167)..(167): Arg or Gln
  MOD_RES (168)..(168): Ser or Pro
  MOD_RES (170)..(170): Val or Thr
  MOD_RES (171)..(171): Ser or Ala
  MOD_RES (176)..(176): Asp or Lys
  MOD_RES (178)..(179): Asp or Ser
  MOD_RES (180)..(180): Arg or Lys
  MOD_RES (189)..(189): Thr or Ile
  MOD_RES (196)..(196): Ser or Glu
  MOD_RES (197)..(197): Ser or Thr
  MOD_RES (200)..(200): Thr or Cys
  MOD_RES (201)..(201): Asp or Glu
  MOD_RES (208)..(208): Asp or Glu
  MOD_RES (209)..(209): Glu or Asn
  MOD_RES (210)..(210): Ile or Val
  MOD_RES (212)..(212): Lys or Val
  MOD_RES (215)..(215): Gly or His
  MOD_RES (216)..(216): Met or Ala
  MOD_RES (218)..(218): Leu or Pro
  MOD_RES (219)..(220): May or may not be present
  MOD_RES (222)..(222): Arg or Ile
  MOD_RES (223)..(223): Asn or Ser
  MOD_RES (224)..(224): Lys or Ala
  MOD_RES (225)..(225): Asn or Lys
  MOD_RES (226)..(226): His or Arg
  MOD_RES (228)..(228): Arg or Lys
  MOD_RES (229)..(229): Glu or Gln
  MOD_RES (230)..(230): Ser or Val
  MOD_RES (235)..(235): Val or Ala
  MOD_RES (244)..(244): Thr or Ser
  MOD_RES (247)..(247): Val or Leu
  MOD_RES (251)..(251): Val or Ile
  MOD_RES (253)..(253): Ile or Leu
  MOD_RES (258)..(258): Thr or Ser
  MOD_RES (262)..(262): Val or Ile
  MOD_RES (266)..(266): Pro or Ser
  MOD_RES (270)..(270): Asn or Gly
  MOD_RES (271)..(271): Thr or Pro
  MOD_RES (272)..(272): Val or Ile
  MOD_RES (276)..(276): Cys or Ala
  MOD_RES (278)..(278): Arg or Ala
  MOD_RES (279)..(279): Tyr or Pro
  MOD_RES (280)..(280): Gly or Asp
  MOD_RES (281)..(281): Thr or Ser
  MOD_RES (285)..(285): Tyr or Phe
  MOD_RES (292)..(292): Thr or Ser
  MOD_RES (293)..(293): Val or Ile
  MOD_RES (294)..(294): Lys or Met
  MOD_RES (295)..(295): Val or Gln
  MOD_RES (298)..(298): Val or Ala
  MOD_RES (301)..(301): Lys or Arg
  MOD_RES (302)..(302): Ala or Thr
  MOD_RES (303)..(303): Phe or Tyr
  MOD_RES (304)..(304): Ile or Met
  MOD_RES (305)..(305): Thr or Asn
  MOD_RES (306)..(306): Glu or Cys
  MOD_RES (307)..(307): Asn or Leu
  MOD_RES (308)..(308): His or Tyr
  MOD_RES (312)..(312): Gly or Asn
  MOD_RES (316)..(316): Ala or Thr
  MOD_RES (317)..(317): Ile or Met
  MOD_RES (321)..(321): Arg or Ser
  MOD_RES (323)..(323): Glu or Asp
  MOD_RES (325)..(325): Ala or Ile
  MOD_RES (329)..(329): Gly or Ala
  MOD_RES (335)..(335): Leu or His
  MOD_RES (336)..(336): Tyr or Leu
  MOD_RES (337)..(337): His or Trp
  MOD_RES (339)..(339): Val or Ile
  MOD_RES (342)..(342): Ser or Glu
  MOD_RES (343)..(343): Thr or Ser
  MOD_RES (344)..(344): Arg or Gln
  MOD_RES (345)..(345): Met or Leu
  MOD_RES (346)..(346): Ile or Val
  MOD_RES (347)..(347): Tyr or Phe
  MOD_RES (351)..(351): May or may not be present
  MOD_RES (353)..(353): Ser or Ala
  MOD_RES (357)..(357): Ser or Cys
  MOD_RES (362)..(362): Ser or Asp
  MOD_RES (364)..(364): Asn or Lys
  MOD_RES (366)..(366): Tyr or Phe
  MOD_RES (372)..(372): Asn or Asp
  MOD_RES (374)..(374): Thr or Ser
  MOD_RES (375)..(375): Val or Ile
  MOD_RES (376)..(376): Ala or Glu
  MOD_RES (379)..(379): Gly or Ser
  MOD_RES (380)..(380): Met or Ile
  MOD_RES (381)..(381): Leu or Met
  MOD_RES (382)..(382): Lys or Arg
  MOD_RES (386)..(386): Val or Ile
  MOD_RES (387)..(387): Phe or Leu
  MOD_RES (388)..(388): Val or Ile
  MOD_RES (389)..(389): Phe or Ile
  MOD_RES (394)..(394): Gln or Pro
  MOD_RES (395)..(395): Asp or Val
  MOD_RES (396)..(396): Ile or Leu
  MOD_RES (397)..(397): Pro or Cys
  MOD_RES (398)..(398): Asp or Thr
  MOD_RES (399)..(399): Gly or Asn
  MOD_RES (400)..(400): Ile or Leu
  MOD_RES (401)..(401): Thr or Asn
  MOD_RES (402)..(402): Thr or Ser
  MOD_RES (403)..(403): Asn or Val
  MOD_RES (404)..(408): May or may not be present
  MOD_RES (409)..(409): Gly or Ser
  MOD_RES (410)..(410): Ile or Pro
  MOD_RES (411)..(411): Leu or Lys
  MOD_RES (415)..(415): Asp or Met
  MOD_RES (417)..(417): Glu or Lys
  MOD_RES (419)..(419): Val or Glu
  MOD_RES (421)..(421): Asn or Val
  MOD_RES (422)..(422): Asn or Pro
  MOD_RES (423)..(423): Cys or Ser
  MOD_RES (424)..(424): Ser or Ala
  MOD_RES (426)..(426): Asn or Gln
  MOD_RES (430)..(430): Asn or Gly
  MOD_RES (431)..(431): Ala or Thr
  MOD_RES (434)..(434): Thr or Ala
  MOD_RES (435)..(435): Ser or Arg
  MOD_RES (441)..(441): Ala or Val
  MOD_RES (449)..(449): Phe or Leu
  MOD_RES (455)..(455): Val or Ile
  MOD_RES (456)..(456): Glu or Lys
  MOD_RES (457)..(457): Thr or Pro
  MOD_RES (458)..(458): Asn or Asp
  MOD_RES (459)..(460): May or may not be present
  MOD_RES (461)..(461): Ala or Gly
  MOD_RES (462)..(462): Ile or Ala
  MOD_RES (463)..(463): Arg or Glu
  MOD_RES (466)..(466): Tyr or Phe
  MOD_RES (467)..(467): Glu or Asp
  MOD_RES (469)..(469): Pro or Lys
  MOD_RES (471)..(471): Thr or Asp
  MOD_RES (479)..(479): Phe or Ile
  MOD_RES (484)..(484): Lys or Arg
  MOD_RES (486)..(486): Leu or Ile
  MOD_RES (487)..(487): Ile or Val
  MOD_RES (494)..(494): Thr or Pro
  MOD_RES (496)..(496): Phe or Leu
  MOD_RES (501)..(501): Cys or Arg
  MOD_RES (502)..(502): Leu or Val
  MOD_RES (503)..(503): Lys or Arg
  MOD_RES (514)..(514): Leu or Ile
  MOD_RES (517)..(517): Ala or Lys
  MOD_RES (518)..(523): May or may not be present
MXXXXXKXXXXXXXRXGXXRGGGXXXXDPFFEXEPKXRRKVXXXDXDIESXDSDXXXXXXXGGGFDEDGXXXXXEXEDEDEFADETXGEKXXRXXXEXLXXXXEXXXRXXEEADNDDDXDEXXXXXXXXXLXXKQXEXSGRXRRXIAXRVXXPXXXDGFXVXVKHRXXVXXVALSXDXXXGFSASKDGXIMHWDVXXGKXXKYIWPSXXXLXSHXXKXREPXXXXXSXXXLALAXSSDGRYLAXGGXDRHXHXWDVRXREHXQAFXGHRXXXSCLXFXXXXSELXSGSFDRXXXXWNXEDXXXXXXXXGHQXEILXXDALXKXRXLTVXRDRTMXXXKXPEXXXXXXRAPAAXSLEXCCFIXDXEXLSGSDXGXXXLWXXXXKKPXXXXKNAHXXXXXXXXXXDNDDEXXXENGXHXPXNXXXXAXSWVXXVAXXRGSDLXASGAGNGXVRLWAXXXXSKXXXPLXXLXLXGFVNSLAXAKSGXFXXAGVGQEXRXGRWGXXXSAQNGVAIHPXRLXDVKEDL

SEQ ID NO: 75 - DNA, 31 bp, Arabidopsis thaliana
ctcaatttgggtcataacgatatctctggtt

SEQ ID NO: 76 - DNA, 29 bp, Artificial Sequence
  Description of Artificial Sequence Synthetic oligonucleotide
ctcaatttgggtcataacgatctctggtt

SEQ ID NO: 77 - DNA, 32 bp, Artificial Sequence
  Description of Artificial Sequence Synthetic oligonucleotide
ctcaatttgggtcataacgatattctctggtt

SEQ ID NO: 78 - DNA, 27 bp, Arabidopsis thaliana
ctcaatttgggtcataacgctctggtt

SEQ ID NO: 79 - DNA, 23 bp, Artificial Sequence
  Description of Artificial Sequence Synthetic oligonucleotide
ctcaatttgggtcataacgggtt

SEQ ID NO: 80 - DNA, 26 bp, Artificial Sequence
  Description of Artificial Sequence Synthetic oligonucleotide
ctcaatttgggtcataacgataggtt

SEQ ID NO: 81 - DNA, 69 bp, Arabidopsis thaliana
caatttgggtcataacgatatctctggttcgattcctgatgaggtaggtgatctaagaggtttaaacat

SEQ ID NO: 82 - DNA, 18 bp, Artificial Sequence
  Description of Artificial Sequence Synthetic oligonucleotide
caatttgggtcataacat

SEQ ID NO: 83 - DNA, 70 bp, Artificial Sequence
  Description of Artificial Sequence Synthetic oligonucleotide
caatttgggtcataacgatatactctggttcgattcctgatgaggtaggtgatctaagaggtttaaacat

SEQ ID NO: 84 - DNA, 70 bp, Artificial Sequence
  Description of Artificial Sequence Synthetic oligonucleotide
caatttgggtcataacgatattctctggttcgattcctgatgaggtaggtgatctaagaggtttaaacat

SEQ ID NO: 85 - DNA, 80 bp, Artificial Sequence
  Description of Artificial Sequence Synthetic oligonucleotide
caaattgacccaatttgggtcataacgatatttctctggttcgattcctgatgaggtaggtgatctaagaggtttaacat

SEQ ID NO: 86 - DNA, 68 bp, Artificial Sequence
  Description of Artificial Sequence Synthetic oligonucleotide
caatttgggtcataacgatactctggttcgattcctgatgaggtaggtgatctaagaggtttaaacat

SEQ ID NO: 87 - DNA, 61 bp, Artificial Sequence
  Description of Artificial Sequence Synthetic oligonucleotide
caatttgggtcatctctggttcgattcctgatgaggtaggtgatctaagaggtttaaacat

SEQ ID NO: 88 - DNA, 38 bp, Unknown
  Description of Unknown Wild-type PDS3 sequence
acataagcctgaccgccgaccatggctggcaaaagtcc

SEQ ID NO: 89 - DNA, 28 bp, Artificial Sequence
  Description of Artificial Sequence Synthetic oligonucleotide
acataagcctgaccgctggcaaaagtcc

SEQ ID NO: 90 - DNA, 36 bp, Artificial Sequence
  Description of Artificial Sequence Synthetic oligonucleotide
acataagcctgaccgccgaccggctggcaaaagtcc

SEQ ID NO: 91 - DNA, 39 bp, Artificial Sequence
  Description of Artificial Sequence Synthetic oligonucleotide
acataagcctgaccgccgaccattggctggcaaaagtcc

SEQ ID NO: 92 - DNA, 37 bp, Artificial Sequence
  Description of Artificial Sequence Synthetic oligonucleotide
acataagcctgaccgccgaccaggctggcaaaagtcc

SEQ ID NO: 93 - DNA, 53 bp, Artificial Sequence
  Description of Artificial Sequence Synthetic oligonucleotide
acataagcctgaccgccgaccaggctgaccgccgactaggctggcaaaagtcc

SEQ ID NO: 94 - DNA, 37 bp, Artificial Sequence
  Description of Artificial Sequence Synthetic oligonucleotide
acataagcctgaccgccgacctggctggcaaaagtcc

SEQ ID NO: 95 - DNA, 47 bp, Artificial Sequence
  Description of Artificial Sequence Synthetic oligonucleotide
acataagcctgaccgccgaccaatagaccaatggctggcaaaagtcc

SEQ ID NO: 96 - DNA, 31 bp, Artificial Sequence
  Description of Artificial Sequence Synthetic oligonucleotide
acataagcctggcccaccatggcaaaagtcc

SEQ ID NO: 97 - DNA, 39 bp, Artificial Sequence
  Description of Artificial Sequence Synthetic oligonucleotide
acataagcctgaccgccgaccataggctggcaaaagtcc

SEQ ID NO: 98 - DNA, 39 bp, Artificial Sequence
  Description of Artificial Sequence Synthetic oligonucleotide
acataagcctgaccgctgaccataggctggcaaaagtcc

SEQ ID NO: 99 - DNA, 35 bp, Solanum lycopersicum; identical to SEQ ID NO: 45

SEQ ID NO: 100 - DNA, 33 bp, Artificial Sequence; identical to SEQ ID NO: 46
  Description of Artificial Sequence Synthetic oligonucleotide

SEQ ID NO: 101 - DNA, 29 bp, Artificial Sequence
  Description of Artificial Sequence Synthetic oligonucleotide
atagtgagtgacggtatcgagggagatgc

SEQ ID NO: 102 - DNA, 29 bp, Artificial Sequence
  Description of Artificial Sequence Synthetic oligonucleotide
atactgagtgacggtatcgagggagatgc

SEQ ID NO: 103 - DNA, 34 bp, Artificial Sequence; identical to SEQ ID NO: 49
  Description of Artificial Sequence Synthetic oligonucleotide

SEQ ID NO: 104 - DNA, 36 bp, Artificial Sequence; identical to SEQ ID NO: 51
  Description of Artificial Sequence Synthetic oligonucleotide

SEQ ID NO: 105 - DNA, 32 bp, Artificial Sequence; identical to SEQ ID NO: 53
  Description of Artificial Sequence Synthetic oligonucleotide

SEQ ID NO: 106 - DNA, 29 bp, Solanum lycopersicum
aaaacaggctcggtttcagcttcggatgt

SEQ ID NO: 107 - DNA, 20 bp, Artificial Sequence
  Description of Artificial Sequence Synthetic oligonucleotide
aaaacaggctcttcggatgt

SEQ ID NO: 108 - DNA, 26 bp, Artificial Sequence
  Description of Artificial Sequence Synthetic oligonucleotide
aaaacaggctcggtttcagcggatgt

SEQ ID NO: 109 - DNA, 27 bp, Artificial Sequence
  Description of Artificial Sequence Synthetic oligonucleotide
aaaacaggctcggtttccttcggatgt

SEQ ID NO: 110 - DNA, 26 bp, Artificial Sequence
  Description of Artificial Sequence Synthetic oligonucleotide
aaaacaggctctgaaacttcggatgt

SEQ ID NO: 111 - DNA, 24 bp, Artificial Sequence
  Description of Artificial Sequence Synthetic oligonucleotide
aaaacaggctcggtcttcggatgt

SEQ ID NO: 112 - DNA, 26 bp, Artificial Sequence
  Description of Artificial Sequence Synthetic oligonucleotide
aaaacaggctcggtttcttcggatgt

SEQ ID NO: 113 - DNA, 25 bp, Artificial Sequence
  Description of Artificial Sequence Synthetic oligonucleotide
aaaacaggctcggttcttcggatgt

SEQ ID NO: 114 - DNA, 30 bp, Artificial Sequence; identical to SEQ ID NO: 68
  Description of Artificial Sequence Synthetic oligonucleotide